(19) United States
(12) Patent Application Publication
(10) Pub. No.: US 2025/0356953 A1
(43) Pub. Date: Nov. 20, 2025

(54) MICROBIAL SENSING AND PREDICTIVE GROWTH MODELING

(71) Applicant: H2OK Innovations Inc., Somerville, MA (US)

(72) Inventors: David LU, Cambridge, MA (US); Richard M. Mariita, Troy, NY (US); Joseph Sanchez, Cambridge, MA (US); Jens HOEFFLIN, Boston, MA (US); Annie LU, Boston, MA (US)

(73) Assignee: H2OK Innovations Inc., Somerville, MA (US)

(21) Appl. No.: 19/210,950

(22) Filed: May 16, 2025

Related U.S. Application Data
(60) Provisional application No. 63/649,668, filed on May 20, 2024.

(51) Int. Cl. G16B 40/20; G16B 40/30

(52) U.S. Cl. CPC G16B 40/20; G16B 40/30

(57) ABSTRACT

Disclosed are systems and methods for microbial sensing and predictive growth modeling. A system can include one or more processors, coupled with memory, to receive genetic information of a microbe in a production system, the genetic information sequenced from a sample taken from the production system. The one or more processors can execute at least one model trained by machine learning using the genetic information to identify the microbe or determine a characteristic of the microbe. The one or more processors can update operation of the production system using the identity of the microbe or the characteristic of the microbe.

### CROSS-REFERENCE TO RELATED PATENT APPLICATION

This application claims the benefit of, and priority to, U.S. Provisional Patent Application No. 63/649,668 filed May 20, 2024, the entirety of which is incorporated by reference herein.

### BACKGROUND

Microbials, such as bacteria, yeasts, or molds, can grow in a production system over time. Production systems may need periodic cleaning or sanitization to remove microbials and prevent microbial growth.

### SUMMARY

At least one aspect of the present disclosure is directed to a system. The system can perform microbial analysis to optimize performance or mitigate risks. The system can include one or more processors, coupled with memory, to receive genetic information of a microbe in a production system, the genetic information sequenced from a sample taken from the production system. The one or more processors can execute at least one model trained by machine learning using the genetic information to identify the microbe or determine a characteristic of the microbe. The one or more processors can update operation of the production system using the identity of the microbe or the characteristic of the microbe.

At least one aspect of the present disclosure is directed to a method. The method can be for managing microbial activity. The method can include receiving, by one or more processors, coupled with memory, genetic information of a microbe in a production system, the genetic information sequenced from a sample taken from the production system. The method can include executing, by the one or more processors, at least one model trained by machine learning using the genetic information to identify the microbe or determine a characteristic of the microbe. The method can include updating, by the one or more processors, operation of the production system using the identity of the microbe or the characteristic of the microbe.

At least one aspect of the present disclosure is directed to one or more storage media. The one or more storage media can store instructions thereon, that, when executed by one or more processors, cause the one or more processors to perform operations. The operations can include receiving genetic information of a microbe in a production system, the genetic information sequenced from a sample taken from the production system. The operations can include executing at least one model trained by machine learning using the genetic information to identify the microbe or determine a characteristic of the microbe. The operations can include updating operation of the production system using the identity of the microbe or the characteristic of the microbe.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. The foregoing information and the following detailed description and drawings include illustrative examples and should not be considered as limiting.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is an example system to perform microbial sensing and predictive growth modeling.

FIG. 2 is an example method of microbial sensing and predictive growth modeling.

FIG. 3 is another example method of microbial sensing and predictive growth modeling.

FIG. 4 is an example architecture of a computing system.

### DETAILED DESCRIPTION

Following below are more detailed descriptions of various concepts related to, and implementations of, methods, apparatuses, and systems of microbial sensing and predictive growth modeling. The various concepts introduced above and discussed in greater detail below may be implemented in any of numerous ways.

Microbiological growth can occur in or on different types of surfaces, media, and/or across different mediums. Understanding or predicting characteristics of microbes (e.g., types of microbes present, their quantification, growth rates, etc.) in or on a sample can be important for many processes. For example, understanding or predicting the characteristics of microbes can allow a production system to ensure product viability, maintain product quality, increase product yields, improve product outcomes, develop effective treatments, monitor environmental health, and advance scientific research. Some testing techniques can rely on collecting samples and culturing the samples. The techniques can involve culturing a microbe on solid or liquid media. However, this culturing can take a long time and can be prone to contamination. For example, this culturing can require days to get results. Furthermore, some types of sequencing, such as Sanger sequencing, take a long time to obtain data, can be expensive, may not be easy to use, and may need a high level of expertise to use. These approaches may not be streamlined for seamless applications.

Because of these issues, many production systems can be designed to operate conservatively for managing microbiological growth. For example, this can include setting short production cycles with frequent stoppages for equipment cleaning to prevent microbiological growth in the equipment. This can also be done through dosing high level of biocides to manage microbiological growth. Furthermore, because the production system may produce a product while a sample is being cultured, the system may not identify batches of the product with a high level of microbes until after the sample culturing and testing is completed. In this regard, if the system identifies that a high level of microbes from the sample, some or all of the product batches may need to be discarded, leading to waste. Furthermore, a system to link product batches with sample tests may be needed to track and identify product batches with a high level of microbe.

Microbial monitoring in production systems can have limitations in both speed and accuracy. For example, culture-based methods could require 24-72 hours to generate results, during which time production would either continue with the risk of contamination or be halted at substantial cost. These methods could also create false negatives, as microorganisms could be viable but non-culturable under standard laboratory conditions. Furthermore, methods could provide limited information about microbe characteristics, growth dynamics, and potential impacts on production processes.

Optical density measurements can be affected by non-microbial particles and require relatively high concentrations of microorganisms before providing reliable detection. Impedance measurement systems may require direct contact with the medium, risking contamination of both the production system and the sensing apparatus. Additionally, sensing technologies may provide only point-in-time measurements with minimal predictive capability, limiting their usefulness for proactive production system management. RF sensing in biological systems could be primarily focused on laboratory applications rather than industrial production environments. The translation of these technologies to real-time monitoring in complex production systems faces challenges related to signal interference, sensitivity limitations, and difficulties in data interpretation. RF sensing methods may lack the integration with complementary sensing modalities and predictive analytics necessary for comprehensive microbial monitoring and management.

Machine learning for microbial analysis could be hindered by limited integration between sensing technologies and analytical platforms. Systems that rely on a single sensing modality could reduce the robustness and comprehensiveness of the analysis. Predictive models could fail to account for the complex interactions between microbial growth dynamics and production system characteristics, leading to inaccurate forecasts and suboptimal intervention strategies.

Methods for scheduling cleaning and sanitization in production systems could rely on fixed time intervals rather than data-driven approaches. This could result in either premature cleaning, causing unnecessary production downtime, or delayed cleaning, allowing microbial populations to reach potentially harmful levels. This lack of adaptive, predictive scheduling capabilities could represent a significant inefficiency in production system operations.

Systems could separate the detection of microbes from the control of production parameters, creating a lag between identification of a potential issue and implementation of corrective measures. This disconnection could lead to product waste, quality issues, and increased production costs. A more integrated approach that directly links detection to automated control adjustments would provide substantial improvements in production efficiency and product quality. Accordingly, there exists a need for improved systems and methods for microbial sensing and predictive growth modeling that overcome these limitations and provide more accurate, timely, and actionable information for production system management.

To solve these and other technical problems, technical solutions of this disclosure can include microbial sensing and predictive growth modeling. For example, a computing system can implement machine learning models or machine learning techniques to determine characteristics of microbes. For example, the computing system can implement one or multiple machine learning models to forecast or predict the growth of a microbe in the production system. The model can identify the presence of microbe, the quantity of microbe, biofilm formation, the type of the microbe, and use the type of the microbe and how rapidly that type of microbe grows to forecast the growth of the microbe using genetic information. The genetic information can be sequenced from a rapid sequencing apparatus, e.g., a nanopore sequencing apparatus. Furthermore, the computing system can utilize one or multiple characteristics of the production system to predict and forecast the growth of the microbe in the production system. For example, the characteristics can include the construction of the production system (e.g., the types of materials used in tanks, the number and types of filters, etc.) or the operating parameters of the production system (e.g., temperature, humidity, or pressure setpoints). The combination of a rapid sequencing apparatus, data analysis, algorithms, and/or machine learning can offer a powerful approach to microbial and viral identification, quantification, and risk prediction. In this regard, by using hardware for rapid genetic sequencing and software to run machine learning techniques, a real-time or near real-time microbe risk prediction and identification system can be implemented.

With the forecasted microbe growth, the production system can be better controlled and operated to increase the amount and quality of product produced by the production system. Furthermore, because the microbe growth is forecasted, the computing system can determine times to clean at, and can schedule cleanings efficiently to avoid unnecessary production system down time. Furthermore, the computing system can update or control the production system to operate with settings (e.g., temperatures, humidities, setpoints, flow rates, etc.) that control the growth of microbes (e.g., either slow the growth of undesirable microbes or increase the growth of desirable microbes such as yeasts). As a result, the microbial sensing and predictive growth modeling described herein can result in higher production yields, and less product waste. The computing system can provide rapid risk evaluation for applications in food inspections or outbreak investigations with improved accuracy. Examples include but are not limited to production planning, cleaning, sanitation, fermentation, etc.

Furthermore, the computing system forecasting and modeling can be used to identity and prevent product spoilage or product impurities. The techniques can result in faster response times, improved accuracy for factory line scenarios, and data driven decision making for contamination/food spoilage control as well as monitoring of beneficial microbes, and catching any competing organisms or harmful viruses for prompt actions. This leads to process savings, recall avoidance, improved yields, and proactive corrections and actions, among other benefits.

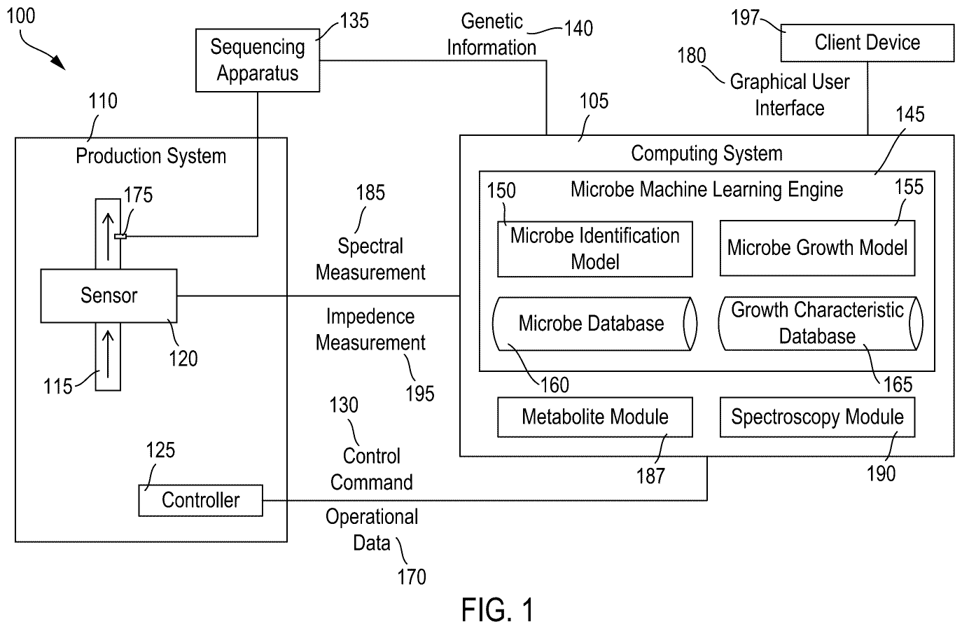

Referring to FIG. 1, among others, an example system 100 to implement machine learning based microbiological predictions is shown. The system 100 can include at least one computing system 105. The computing system 105 can be a local gateway, a local controller, an on-premises computing system, an off-premises computing system, a server, a server system, a cloud computing system, or any other data processing system, apparatus, or device. The computing system 105 can be a computer or data analysis device. The system 100 can be implemented for a production environment. The computing system 105 can be communicably coupled with at least one production system 110. The computing system 105 can be located on-premises with the production system 110, or may be off-premises and remote from the production system 110. The computing system 105 can be integrated with, or a component of, the production system 110, or may be a separate component.

The production system 110 can produce at least one product. The production system 110 can be a system to manufacture or produce a product, such as a food product. The production system 110 can manufacture or produce a food, a drink, or any other substance. The production system 110 can manufacture a condiment (e.g., ketchup, mayonnaise, vegetable oil, olive oil, mustard), a dessert (e.g., ice cream, sherbet, yogurt), a food (e.g., yogurt, cream cheese), a drink (e.g., a soft drink, a cola, wine, beer, liquor, an energy drink, vitamins, coffee, purified water, milk), a chemical, an ingredient, an additive, a flavor, a fragrance, an oil, a pharmaceutical, a cleaning product, a hygiene product (e.g., a shampoo, a toothpaste, a soap, a mouth wash). The product can be a solid, e.g., a pharmaceutical. The product can be a gas. The product can be a powder. The product can be a liquid, a solid, a gel, a semi-liquid, or any other composition. The production system 110 can receive one or multiple ingredients, mix the ingredients, emulsify the ingredients, cook the ingredients, cool the ingredients, boil the ingredients, or perform a variety of other production steps to produce the product. The production system 110 can include, but is not limited to, mixing equipment, heating equipment, cooling equipment, tanks, reactors, pit, pond, lake, reservoir, ocean, container, pipe, river, or presses.

The production system 110 can include at least one line, conduit tank, or product holding apparatus 115. The apparatus 115 can be a conduit, pipe, cavity, tank, canal, or other area carrying a liquid, solid, gas, gel, etc. such as the product, ingredients to make the product. The apparatus 115 can be a line which moves liquid, or can be any other apparatus that holds a liquid. The apparatus 115 can be a line carrying liquids into the production system 110 or carrying liquids out of the production system 110. The apparatus 115 can carry waste product out of the production system 110 to be disposed. The product or material of the line 115 can be at least partially mixed or suspended in water or non-water material (e.g., a cleaning product, a sanitizer, a product transfer). A sensor 120 (e.g., a spectral sensor or impedance sensor) can be disposed or submerged at least partially in a fluid within the line, tank, or fluid holding apparatus 115. For example, the sensor 120 can be dropped into a tank of the production system 110 and at least partially submerged within a liquid of the tank. In some implementations, the system 100 can be applied to a non-production system, e.g., a vehicle or apparatus that carries, moves, or transports a product. For example, the system 100 can be implemented for a truck that carries a product such as a tanker truck, a rail car, a transport vessel, a container, a mixing truck, etc. Furthermore, the system 100 can be implemented at a water treatment plant, in a cleaning filter, etc.

The production system 110 can include at least one controller 125. The controller 125 can be a programmable logic controller, a microprocessor, a computer, an inverter, a distributed control system (DCS), a programmable logic controller (PLC), a building management system (BMS), a supervisory control and data acquisition (SCADA) system or any other device that can control actuators of the production system 110 to control the production of the product. For example, the controller 125 can open or close valves based on the control command 130. The controller 125 can start or stop a fan, or control the speed of a fan, based on the control command 130. The controller 125 can control heating devices or cooling systems to meet a temperature (e.g., increasing or decreasing temperature), based on the control command 130. The controller 125 can start or stop a mixer by operating a motor or set a speed of the mixer, based on the control command 130.

The system 100 can include at least one sequencing apparatus 135. The sequencing apparatus 135 can generate or determine genetic information 140 from a sample taken from the production system 110. The sequencing apparatus 135 can provide rapid DNA or RNA extraction from a sample. The genetic information 140 can be genetic information of at least one microbe located in or growing within the production system 110. The microbe can be a fungi, an algae, a protist, a bacteria, an archaea, etc. The microbe can be used in production of a product, e.g., a yeast or probiotic, or can be a harmful or disease causing microbe (e.g., legionella, salmonella, e-coli). The sequencing apparatus 135 can sequence DNA and/or RNA information. The sequencing apparatus 135 can allow for swift or rapid genetic sequencing for identification of microbes or viruses in a sample. This quick sequencing can allow rapid or quick risk assessment and/or real-time control or operation of the production system 110 using the genetic information 140. The sequencing apparatus 135 can be a portable or stationary apparatus that provides rapid or real-time DNA and/or RNA sequencing. The sequencing apparatus 135 can be a nanopore device. The DNA or RNA can be sequenced by the sequencing apparatus 135, which can include a microfluidic chip with nanopores, electrical connections, power, and data transfer capability. For example, the sequencing apparatus 135 can be an OXFORD NANOPORE MINION. In some implementations, microbial taxonomy sequencing can be performed via grab sampling through a benchtop unit, e.g., to understand all types and concentrations of initial microbial loads. Sequencing can include sample collection using a sterile grab sampling unit from the food production line 115.

The sequencing apparatus 135 can be a sample collection unit (e.g., handheld unit or a part of a system made out of stainless steel, plastic, etc.) with a specific configuration for collecting a sample from the equipment, vessel, or sample line 115 without contaminating samples. The sequencing apparatus 135 can include an extraction kit portion that includes reagents such as enzymes, buffers, etc., with specific compositions for isolating microbial DNA or RNA from the collected sample.

The production system 110 can include at least one port 175. The port 175 can be coupled with, or integrated into, the apparatus 115. A sample of the production can be taken from or through the port 175 and provided to the sequencing apparatus 135 for genetic sequencing. The port 175 can be located in a high risk area or component of the production system 110 to take samples from areas where microbes are likely to grow. For example, the port 175 can provide samples from processing tanks, transfer lines, holding tanks, vulnerable connections, and/or dead legs (e.g., areas with low flow). The port 175 can provide samples from upstream of a location of interest, downstream of a location of interest, or at the location of interest. The samples can be collected in-line or scooped, depending on their type and ease of access. The sample can be sample of a liquid, surface, powders, product produced by the production system 110, an ingredient used by the production system 110, or an intermediate material produced by the production system 110 and used to create a product. Samples can be taken when the production system 110 is on or off, taken inline, taken by swabs, taken by scoops, taken by filtration, or taken by any other technique. The samples can be taken from a food production system, a beverage production system, a cosmetics production system, a solids production system, a powders production system, a pharmaceutical production system, a chemical production system, a liquid production system, a fluid production system, a gas production system, a gel production system, or any other type of production system.

The computing system 105 can be communicably coupled with the sequencing apparatus 135 via at least one network, communication channel, communication bus, wired medium, wireless network, etc. The computing system 105 can be connected to the sequencing apparatus 135 via at least one wired or wireless connection. The computing system 105 can receive the genetic information 140 from the sequencing apparatus 135. The computing system 105 can store the received genetic information 140 in at least one memory device, storage device, or database. The computing system 105 can process the stored genetic information through a model or machine learning model that identifies the presence and abundance of certain microorganisms or viruses.

The computing system 105 can include at least one microbe machine learning engine 145. The machine learning engine 145 can implement machine learning techniques, such as artificial intelligence. The machine learning techniques can include supervised, unsupervised, or semi-supervised techniques. The microbe machine learning engine 145 can generate data for use in monitoring and/or predicting microbial communities during a production process performed by the production system 110, e.g., such as fermentation for beer manufacturing, pharmaceutical, small molecule, probiotic, and other supplement production, as well as fermentation control monitoring for beverages (e.g., wine and spirits, beer, kombucha), sauces, and foods (e.g., vinegar, soy sauce, cheese, yogurt, MSG chicken bouillon). The microbe machine learning engine 145 can execute at least one model trained using a machine learning technique using the genetic information 140, information of a microbe database 160, or information of a growth characteristic database 165. For example, the engine 145 can execute at least one microbe identification model 150 to identify the microbe. The model 150 can identify the type, genus, species, or taxonomy of the microbe or contaminants from the extracted genetic information 140. The engine 145 can execute at least one microbe growth model 155 to forecast the growth of the identified microbe. The engine 145 can determine population dynamics of a microbe. The microbe identification model 150 can identify microbes anonymously or generically. For example, the microbe identification model 150 can identify one or multiple distinct microbes in a sample without cataloging the type, genus, species, or taxonomy of the microbe. For example, the microbe identification model 150 can identify a generic microbe 1, microbe 2, microbe 3, etc. For example, the microbe identification model 150 could implement a supervised machine learning model to identify a specific type, genus, species, or taxonomy of a microbe, or an unsupervised machine learning model to identify a generic microbe, e.g., microbe A, microbe B, microbe C, etc.

The engine 145 can include models 150 or 155 that are pre-trained model of a software program residing on the computing system 105, on a network, on-premises, off-premises, or on the cloud. The engine 145 can run a single complete model deployed to the engine 145, or can run one or multiple partial models deployed to the engine 145. A cloud platform or server system can deploy additional models to the engine 145 over time and updates to the model can be made over time with improved advancements. The cloud or server system can deploy multiple models for multiple microbes, viruses, and genomic material, to the engine 145.

The models 150 and/or 155 can be pre-trained models, e.g., trained via a supervised or semi-supervised machine learning technique. For example, the models 150 and/or 155 can be neural networks (e.g., sparse or dense networks, recurrent neural networks, sequence neural networks, long-short term neural network, etc.), decision trees, Naïve Bayes, regression, etc. The models 150 and/or 155 can be trained via training data which can be or include information of the microbe database 160 or the growth characteristic database 165. The training data can indicate RNA or DNA information for different microbe types. The training data can indicate measured microbe growth under different environmental conditions or for different production system characteristics. The engine 145 can implement a training technique or training algorithm based on microbial data and/or environmental characteristics. The engine 145 can train the model 150 and/or 155 using a loss function and updating weights or parameters of the models 150 or 155 via backpropagation and gradient descent (or stochastic gradient descent, nonlinear conjugate gradient, Levenberg-Marquardt algorithm, etc.). The models 150 and/or 155 can be unsupervised machine learning models. The models 150 or 155 can be algorithms or processes that are executed by the engine 145. For example, the unsupervised technique can be a cluster analysis or association analysis, e.g., k-means clustering, k-medoids clustering, hierarchical clustering, hidden Markov model clustering, etc. The models 150 or 155 can be Large Language Models (LLM), State Space Models (SSM), or derivative or variants of them. The models 150 or 155 can output information on current or predicted microbe, chemistry, characteristics, quality, control action, command, alert, or more. The models 150 or 155 can be an algorithm.

In some implementations, the engine 145 trains the models 150 or 155 based on genetic information 140 collected over time from the production system 110 and operational data 170 collected over time from the production system 110. In some implementations, the engine 145 can continuously or repeatedly receive genetic information 140 and operational data 170, and continuously or repeatedly identify microbes or predict microbe growth using the models 150 or 155. In some implementations, the sequencing apparatus 135 can perform in-line genetic taxonomy sequencing, and continuously or iteratively provide genetic information 140 to the computing system 105 for the engine 145 to run on. In some implementations, based on historical data, the engine 145 can retrain or tune the models 150 and/or 155. In some implementation, the samples taken and sequenced can be inoculated.

Furthermore, the engine 145 can execute at least one microbe growth model 155 to predict, forecast, or determine the growth of the microbe into the future. For example, the model 155 can forecast the amount, concentration, quantity, or level of the microbe that will be present in the production system 110 at multiple time steps into the future. The model 155 can use microbe taxonomy data (e.g., sequenced microbial and genome results) and/or an initial or measured microbial load to predict future microbial growth. The model 155 can execute based on operational data 170 that describes characteristics or an environment of the production system 110. The model 155 can predict the growth of the microbes into the future based on the environment of the production system 110 that the microbes will be growing in. The model 155 can combine all collected data or a subset of collected data to predict potential microbe growth and associated risk, which feeds into the alert system.

The operational data 170 can indicate characteristics of the production system 110 where the sample was taken. For example, the characteristics of the production system 110 can include the design of the production system 110 (e.g., types of equipment, pumps, materials that the production system 110 is manufactured from). For example, the characteristics of the production system 110 can include the medium or material which the sample was taken (e.g., water, mayonnaise, dairy, powders, solids, slurries, beer, drinks, sauces, foods, pharmaceuticals, chemicals, fuels, fermented products, plastics, gasses, natural waterbodies, cooling liquids, etc.) or what other types of materials are present with the microbes. For example, the characteristics of the production system 110 can include operating settings of the production system 110 (e.g., temperature setpoints, timer lengths, humidity setpoints, etc.), sensor measurements (e.g., in-line sensor measurements monitoring environmental parameters such as chemical composition, pH, salinity, temperature, chemistry, flow rate, turbidity, nutrient availability, biofilms, total dissolved solids (TDS), conductivity, impedance spectroscopy data 195, light spectroscopy data 185, RF data, image data, video data, optical data, alcohol level data, ultraviolet transmittance or transmission (UVT), etc.).

For example, the characteristics of the production system 110 can include a maintenance history of the production system 110. For example, the characteristics of the production system 110 can include a cleaning history of the production system 110. For example, the characteristics of the production system 110 can include indications of pump operations (e.g., variable frequency drive (VFD) information temperature in the pumping sample of the vessel), line operations (e.g., whether a process line is running or not), etc. The operational data 170 can indicate a present or scheduled characteristic of the production system 110. For example, the operational data 170 can indicate operating temperatures for one or multiple batches or time steps into the future. The operational data 170 can include additional data to be fed into the model 155, e.g., the environment or vessel containing the sample. For example, the information about the vessel containing the sample can be its shape and surface area, the material it is made up of, its surface treatment and characteristics, information of areas prone to microbe growth in the vessel, etc.

The machine learning engine 145 can monitor the presence of viruses that can infect beneficial bacteria (e.g., phages) and yeasts/fungi reported in fermented products such as milks, sausages, vegetables, wine, sourdough, and/or beans. Furthermore, the machine learning engine 145 can help detect viruses, such as human noroviruses, rotavirus, and hepatitis virus which may be present in fermented products.

The microbe machine learning engine 145 can include a microbe database 160. The microbe database 160 can store various genetic data classified for various different types of microbes. For example, the database 160 can store reference DNA or RNA sequences for different classes of microbes, yeasts, fungi, or viruses. The microbe database 160 can be a digital database stored on the computer, connected to a network with external reference DNA or RNA sequences (such as GenBank etc.) in formats such as FASTA and GenBank. The reference DNA or RNA can be received from GenBank, RefSeq, DNA Data Bank of Japan (DDBJ), European Nucleotide Archive (ENA), EMBL Nucleotide Sequence Database, or any other reference databases. For example, the microbe database 160 can store DNA or RNA information for each of multiple different types of microbes. The microbe database 160 can be a key-value database, a RDMS database, a SQL database, a noSQL database, a vector database, a graph database, and/or any other type of database. The microbe identification model 150 can execute at least one matching or pattern identification algorithm using the genetic information 140 and the microbe database 160. In some implementations, the microbe identification model 150 can be trained by a machine learning technique using the microbe database 160.

The growth characteristic database 165 can store growth characteristic data for microbes. The growth characteristic database 165 can store data that indicates growth rates for various types of microbes. The database 165 can indicate potential risks for various microbes, and/or the levels, amounts, or concentrations at which microbes may be dangers to consumers of the products produced by the production system 110. The growth characteristic database 165 can indicate the growth rates for various types of microbes according to various environmental characteristics, e.g., temperature, humidity, pressure, pH, salinity, alcohol level, oxygen level, light levels, etc. The growth characteristics database 165 can include information on growth characteristics and potential risks of the microbes, or to monitor the viability and abundance of specific beneficial strains during production.

The growth characteristic database 165 can be a key-value database, a RDMS database, a SQL database, a noSQL database, a vector database, a graph database, and/or any other type of database. The growth characteristic database 165 can use the growth rates indicated by the growth characteristic database 165 for a microbe identified by the microbe identification model 150 to predict the amount of the microbe in the production system 110 one or multiple timesteps into the future. In some implementations, the microbe identification model 150 can be trained by a machine learning technique using the growth characteristic microbe database 165.

In some implementations, the microbe database 160 and/or the growth characteristic database 165 are part of the computing system 105, or are separate from the computing system 105. The separate databases 160 and 165 can be connected with the computing system 105 via a local network or external network for potential database access or data storage depending on the specific configuration or requirements for a client.

The computing system 105 can use the identified microbes or forecasted microbe growth to generate control commands 130. The control commands 130 can be changes or adjustments to operating parameters of the production system 110 that can be implemented by the controller 125. For example, the control commands 130 can lengthen or shorten baking times, increasing or decreasing temperature, shorten or lengthen mixing times, shorten or lengthen fermentation times, stop or start fermentation, add a material, etc. The control commands 130 can raise or lower temperature, humidity, or pressure of the production system 110. The control commands 130 can be signals, values, messages, data frames, settings, setpoints, etc. The computing system 105 can change the control parameters or controls responsive to identifying the presence, characteristic of a microbe (e.g. biofilm formation state), or a particular quantity level of the microorganism being reached.

The computing system 105 can generate the control commands 130 using the identified microbe and/or the predicted growth of the microbe. For example, the computing system 105 can generate the control commands 130 to control the growth of the microbe. The computing system 105 can generate the control commands 130 to maximize or increase the product. For example, the computing system 105 can generate control commands 130 that slow the growth of the microbe, and allow the production system 110 to run for an extended period of time before needing a cleaning.

In some implementations, if the microbe is a desirable microbe, such as a yeast that may be needed for fermenting a product, the computing system 105 can use the forecast of the microbe to generate a control command 130 that increases or speeds up the growth of the microbe. For example, the computing system 105 can generate control commands 130 to optimize growth conditions for beneficial microbes. For example, the production system 110 can make fermented products leveraging the microbe identification and predicted growth by the engine 145 to monitor the growth rates and viability and abundance of specific beneficial bacteria or fungal strains during production for better controlled manufacturing processes. This can ensure consistent quality and efficacy of the final fermented product. This can be achievable by not only monitoring the health of beneficial bacteria or fungi/yeast and optimizing growth conditions, but for early/rapid detection of microbial competitors.

In some implementations, the computing system 105 can store a list of desirable or good microbes (e.g., yeasts, probiotics, etc.) and a list of undesirable or bad microbes (e.g., legionella, salmonella, e-coli). The list can be specific to the product that the production system 110 is producing. The bad microbes can be microbes that spoil a product, taint a product, or are dangerous to a consumer. The good microbes can be microbes needed for the product, such as yeast for an alcohol fermentation process, yeast, for a bread baking process, or a probiotic for a supplement, food, or drink. The computing system 105 can compare the identified microbes determined by the microbe identification model 150 to the lists.

Responsive to identifying the microbe on the bad or undesirable list, the computing system 105 can generate at least one control command 130 that slows, stops, or limits the growth of the microbe in the production system 110. The control command 130 generated by the computing system 105 can update operation of the production system 110 to slow growth of the microbe responsive to the determination that the microbe is classified as the dangerous microbe. The computing system 105 can execute the microbe growth model 155 one or multiple times with different control commands 130 to identify a control command that slows the growth of the dangerous microbe. The computing system 105 can run the model 155 multiple times to identify an optimal control command 130 that that results in the slowest growth or maximizes the product production without allowing the microbe population size to reach a particular threshold level. The threshold level can be indicated by the list, e.g., the list can indicate maximum allowable levels of dangerous microbes. For example, the computing system 105 can analyze a signal (e.g., the spectral measurement 185 or the impedance measurement 195, optical, RF, image, camera, video, pH, conductivity, salinity, dissolved oxygen, alcohol concentration, temperature, flow) to determine a population of a microbe in a product, compare the concentration level to a threshold, and modify operation for the production system 110 to divert or treat product when the concentration meets or exceeds the threshold.

In some implementations, the computing system 105 can compare the identified microbe to a good or beneficial microbes list, and determine that the identified microbe is on the good microbes list. For example, the computing system 105 can identify that the microbe is a desirable yeast for producing a product with. Responsive to identifying the microbe on the good or desirable list, the computing system 105 can generate at least one control command 130 that controls the growth microbe to a level in the production system (e.g., speeds up, slows down, increases, or decreases growth of the microbe to the level). The control command 130 generated by the computing system 105 can update operation of the production system 110 to control growth of the microbe to the level responsive to the determination that the microbe is classified as the good microbe. The computing system 105 can execute the microbe growth model 155 one or multiple times with different control commands 130 to identify a control command that controls the growth of the good microbe to the level. The computing system 105 can run the model 155 multiple times to identify an optimal control command 130 that identifies a command 130 that results in the fastest growth of the microbe to the level or maximizes the product production.

The microbe identification and growth prediction can be used by the computing system 105 to control wastewater treatment, can be used in antibiotic resistance monitoring to rapidly identify and track the spread of antibiotic-resistant bacteria in healthcare settings, used in laboratories and other settings in remote and urban remote areas to gather crucial microbial information (e.g., taxonomic, population dynamics and functional clues) for implementing targeted infection control measures and preventing outbreaks, environmental monitoring of soil, water and air for gaining insights into environmental health, as well as monitoring bioremediation efforts for oil spills and other environmental contamination. Further, the microbe identification and growth prediction can be used for monitoring the growth of cells like meat, for early detection of potential contaminants allowing for swift intervention to prevent spoilage or contamination of the lab grown meat product or cell cultures or enzymes, and monitoring the production of pharmaceuticals to ensure clean conditions are not compromised through the identification of microbial and viral DNA sequences and loading that can be combined with machine learning models and prediction models. Another use of the machine learning engine 145 can be in monitoring of biofouling and bio growth in liquids and on surfaces (e.g., cooling water, cooling fluid, environmental water, aquaculture) which has applicability in data centers (AI and cloud computing industry) and other industries that require significant cooling to function properly.

In some implementations, the computing system 105 can distinguish between living and nonliving (dead) microorganisms via inoculation. The computing system 105 can compare amounts of a microbe before and after inoculation. The computing system 105 can receive results of inoculating the sample and conduct a sequence of the initial sample to understand a baseline presence of microbial quantity. Then, a set amount of time can be given for the inoculated sample to replicate. After the set amount of time, another sequence of the inoculated sample can be taken to determine the identity and quantification of microorganisms in the sample. The computing system 105 can receive the amounts of the microbes, and use a growth model or comparison to determine the types and amounts of living microorganisms in the original sample. The samples can be inoculated on growth media such as R2A, Nutrient Agar, Tryptic Soy Agar (TSA), MacConkey Agar (MAC), for a duration of time from which the sequencing can be conducted to allow for lower failure rates or for machine learning training purposes. TSA, R2A and MAC cam be incubated at around 35-37° C., and at room temperature (20-25° C.). Sabouraud Dextrose Agar (SDA) can be used to culture fungal contaminants with incubation being done at 25° C. and at 30° C. Incubation can be performed 24-72 hours, or as desired. In some implementations, the microbe can execute the microbe growth model 155 or the microbe identification model 150 with the indication of which microbes are dead or alive.

The computing system 105 can use the growth modeling performed by the engine 145 to raise alerts, pass signals, or recommend actions to be taken by a user. Furthermore, by predicting growth, better planning and proactive measures can be taken by the computing system 105. The computing system 105 can be coupled with a client device 197. The client device 197 can be a laptop computer, a desktop computer, a smartphone, a tablet, etc. The client device 197 can be a device for a user or customer to interact with the computing system 105 and view at least one graphical user interface 180 on a display of the client device 197. The graphical user interface 180 can display alerts that a microbe has reached a particular quantity, an alert to clean in place (CIP), an alert to clean, an alert to stop fermentation, etc. The graphical user interface 180 can show a predicted time when the production system 110 will need cleaning to prevent the microbe quantity from reaching the particular quantity. The graphical user interface 180 can include a progress bar that showcases microbe load and growth risk in line to identify in data-driven approach when to clean in place (CIP), sanitize, or treat the system or vessel containing the sample with the microbe. The graphical user interface 180 can include allow a user to input operating parameters, and given the inputted operation parameters, the engine 145 can generate projected microbe growth and when the next CIP, sanitization, or system treatment should be performed, which can be displayed in the graphical user interface 180. Similarly, the engine 145 can predict and model a fermentation process, the results, and when fermentation is complete can be displayed in the graphical user interface 180.

As an example, the computing system 105 can test for microbe contamination in food, beverages, and consumables. The computing system 105 can determine whether microbial loading is below a certain concentration in these materials to ensure human or consumer health. For example, the computing system 105 can receive measurements to determine microbial loading into and out of a tank to detect when the perform cleaning or changeovers. Based on the microbial activity, the computing system 105 can prevent quality challenges, and better control when to start, stop, and clean processing equipment or whether or not an ingredient or material can still be used. As another example, the computing system 105 can monitor or control microbial activity for fermentation. For example, contamination of competitive or unwanted bacteria or fungi within the initial fermentation ingredients or colony can lead to stopped or stuck fermentation, spoiled product, or unwanted results. The computing system 105 can use determined microbial activity and the byproducts of microbial activity control a fermentation process for greater efficacy. As another example, the computing system 105 can implement microbial monitoring and forecasting for monitoring and characterizing biofilm on a surface. Biofilm can cause contamination risk, corrode and damage the surface, and lead to health risks. The computing system 105 can identify, quantify, and qualify biofilm growth for control, treatment, and prevention. As another example, the computing system 105 can use microbial activity and its byproducts in the measurement of biological reactions and biological growth, as well as disease screening.

The computing system 105 can include at least one metabolite module 187. The metabolite module 187 can integrate with chemical sensing of secondary microbial metabolites. The metabolite module 187 can receive an indication of a metabolite determined through chemical or spectral sensing, and identify a microbe that produced the metabolite and whether the metabolite is alive or dead. Bacteria and mold can produce secondary metabolites that cause spoilage and off-flavors. Secondary metabolites and their load can be identified by the metabolite module 187 for identifying microbial level and risk through functional analyses. Example secondary metabolites can include organic acids (e.g., lactic acid, acetic acid, butyric acid), enzymes, bacteriocins, pigments.

In some implementations, aseptic techniques can be used to obtain samples of metabolites from the production system 110. Samples can be in liquid, solid, or gaseous form. Samples can be stored at appropriate temperature and environmental conditions, or processed immediately. Sample preparation can include extraction of metabolites from samples, and or filtration to remove particulates and cell debris. Identification and quantification of secondary metabolites can be performed but not limited to using High-Performance Liquid Chromatography (HPLC), Gas Chromatography-Mass Spectrometry (GC-MS), or using biosensors. Biosensors can involve biological components (enzymes, antibodies) that react specifically with target metabolites, generating a measurable signal. The generated data, in the form of peaks and spectra, can be used for identification of metabolites by matching against reference databases or models by the metabolite module 187.

The metabolite module 187 can perform spectral analysis based on spectral measurements 185 received from sensors 120. The metabolite module 187 can include spectral libraries, cheminformatics software, and/or metabolic pathway databases. The metabolite module 187 can implement machine learning for analyzing complex datasets related to secondary metabolites. The metabolite module 187 can identify metabolites associated with both beneficial microbes (e.g., bacteria or fungi) as well as contaminants (for instance lactic acid from lactic acid producing bacteria). Because the metabolite module 187 uses metabolites to detect microbes, the approach can be non-destructive (e.g., does not require cell lysis), and can implement multiplexing, simultaneous detection of several metabolites, e.g., many types of metabolites can be detected at same time, whether from the beneficial microbes, or contaminants. The metabolite module 187 can implement metabolite sensing which can have broad applications, as it can be applied to gasses liquids (e.g., food, beverages, liquid media with growth cells), and/or solids (e.g., food, soils). DNA sequencing may not be able to discriminate between active and inactive/dead microbes. Future alternatives include advanced biosensors that offer rapid detection capabilities, microfluidic devices that can be used in processing samples, detection and metabolite analysis, further enabling miniaturized and automated microbial monitoring systems.

The computing system 105 can include at least one spectroscopy module 190. The spectroscopy module 190 can use impedance spectroscopy or spectral sensing to analyze microbe concentration in line. The spectroscopy module 190 can receive at least one measurement 195 from at least one sensor 120. The impedance measurement 195 can be an impedance measurement or a spectrum of impedances, amplitudes, or phases for various frequencies. For example, the sensor 120 can include one, two, or more electrodes disposed within a material of the production system 110. The geometries of the electrodes inserted into the liquid, gel, or solid medium can be varied. For example, the sensor 120 can include a first electrode and a second electrode separated by a distance. Furthermore, an impedance spectral sensor 120 can be placed in a line 115 in a non-sanitary or sanitary configuration. Multiple impedance spectral sensors 120 can be disposed or placed in a production system 110. For example, one impedance sensor 120 can be placed at the beginning of a system or equipment, and one at the end of a system or equipment like a processing line. A first electrode can emit or produce a signal in the product that the second electrode can sense. The product can be disposed between the first and second electrodes.

The spectroscopy module 190 can cause the first electrode to apply a signal to a sample, and receive a signal from the second electrode separated from the first electrode. The spectroscopy module 190 can sweep a frequency applied to the first electrode. By rapidly sweeping through the applied frequencies, and measuring the signal received from the second electrode, the spectroscopy module 190 can build an impedance spectrum of the sample versus frequency. In some implementations, the frequency, voltage, or phase of electricity applied to the first electrode or measured by the second electrode can vary and be recorded, measured, or determined.

The spectroscopy module 190 can distinguish between different species of microorganisms, fungi, or bacteria based on different effects that the microorganisms, fungi, or bacteria have on the impedance measurement 195 of the sample they are present in. Furthermore, the spectroscopy module 190 can distinguish, using the impedance measurement 195, between living microbes and dead microbes based on the effects on impedance the microbes have on the sample. The spectroscopy module 190 can identify differing measured impedances 195 via impedance sensors 120, and provide the sensed impedances 195 to the models 150 or 155 along with other inputs to identify the presence of a species of microbes, the quantity of the microbe, and the predicted growth of the microbe.

The spectroscopy module 190 can implement impedance spectroscopy to continuously monitor a microbe concentration in a production system 110 or production line 115, the formation of biofilm, the presence of biofouling, the presence of scaling, minerals, or material buildup on a surface. The spectroscopy module 190 can receive impedance measurements 195 and feed the impedance measurements 195 to the models 150 or 155 for microbe monitoring and predictive modeling or in place of the genomic sequencing data for understanding microbe identification and quantification.

The spectroscopy module 190 can implement impedance spectroscopy to detect subtle changes in microbe concentration for preventive notifications. The spectroscopy module 190 can implement impedance spectroscopy to identify changes in chemistry and materials in the samples. The spectroscopy module 190 can implement impedance spectroscopy to perform quality and material classification. The spectroscopy module 190 can use the impedance measurement 195 to measure chemistry and materials in a sample by feeding the measurement 195 into a predictive model 155 or algorithm to predict process outcomes and for generating alerts or updating control commands 130. For example, the computing system 105 can use the impedance measurements 195 to forecast microbe growth for monitoring or optimization for a chemical reaction, fermentation process, digestion process, changeovers, or quality control.

In some implementations, the spectroscopy module 190 can compare the signal applied to the first electrode to the signal measured by the second electrode to identify changes in amplitude, changes in phase, or changes in frequency, and these measurements can be used by the module 190 to identify microbes, predict microbe growth, or classify microbes as alive or dead. For example, the spectroscopy module 190 can use at least one receiving electrode to measure the impedance and phase shift induced by the media the electricity passes through. Microbes, bacterial colonies, and other contaminants can have distinctive effects on these electrical characteristics of the media. The spectroscopy module 190 can be electrically coupled with the receiving electrodes to determine the changes in amplitude, phase, or impedance. The computing system can then pass the data through a series of data analysis models or algorithms 150 or 155 to predict and calculate which contaminants or bacterial species are present and their quantity, the presence of the contaminant or microbe, or the type of contaminant or microbe.

Furthermore, the module 190 can use the impedance spectral measurements 195 to measure microbial metabolites as well as chemicals consumed and created by microorganisms. The computing system 105 can use the determined metabolites or chemicals to predict microbe growth and change. The computing system 105 or the spectroscopy module 190 can implement one or multiple data analysis modules to analyze the impedance, capacitance, and spectral data looking at the data in different manners. For example, the computing system 105 can include multiple models deployed to analyze different aspects of the spectrum 195 as well as changes in the spectrum 195 for different organisms, microorganisms, viruses, chemicals and compounds consumed and created by them, the metabolites created by them, as well as the chemistries and composition of a sample. Impedance spectral measurements 195 can also include RF measurements.

The module 190 can implement models to analyze a current impedance measurement 195 or spectral measurement 185, a timeseries of measurements 185 or 195, or changes in timeseries data to predict changes and future results. The data and models can also be applied to identify process effectiveness and material quality. For example, the data and models can identify how well a material is cooked (e.g., digestion of soybeans, corn, wheat, etc.) or processed, the activities, active ingredients, and chemistries of a material, transitions of one material or state to another (e.g., product changcovers, material pushout, sanitation and cleaning), as well as reaction, and fermentation rate tracking and predicting. The results can be output continuously in real-time via the graphical user interface 180, as well as logged and stored by the computing system 105. The spectroscopy module 190 can be built and tuned on historical data and collected data for a specific production system 110.

In some implementations, one impedance sensor 120 can be placed at the beginning of a system or equipment, and one impedance sensor 120 positioned at the end of a system or equipment like a processing line. The spectroscopy module 190 can compare results between multiple sensors 120 to identify the effectiveness or completion of a process. The computing system 105 can use the comparison to initiate a state change, changeover, reaction, or cleaning of the system, such as CIP. The module 190 can input data measurements 195 from multiple sensors 120 into a machine-learning model that can output control or alert information or output useful data and results. The model can be deployed in the sensors 120 themselves, on the same network as the sensors 120, on the computing system 105, or on an external network. Multiple models can be deployed to analyze changes in impedance spectral data 195 from multiple sensors 120 in different ways to identify different parameters or process outcomes.

The spectroscopy module 190 can implement impedance spectroscopy for chemicals, pharmaceuticals, foods, snacks, candies, sauces, soy sauce, vinegar, MSG chicken bouillon, beverages, beer, wine, spirits, cosmetics, fuels, CPG, home care, personal care, powders, flavors, fragrances, and dairy production and fields as well as waste or wastewater treatment. The spectroscopy module 190 can implement impedance spectroscopy for monitoring bio-growth in a system and material buildup and biofilm formation on the surface of a material in industries such as but not limited to cooling and boilers, applicable in many industries such as data centers, power plants, steel making, mills, and buildings. The samples can be liquids, fluids, powders, solids, plasmas, and gasses.

The spectroscopy module 190 can implement spectral analysis using spectral measurements 185, in some implementations. The spectroscopy module 190 can provide real-time continuous monitoring of microbe concentration in the production system 110. The spectroscopy module 190 can scale the number of spectra measurements 185 collected and processed for various numbers of sensors 120. The spectroscopy module 190 can implement spectral sensing to detect subtle changes in microbe concentration for preventive notifications.

The sensor 120 can include at least one light source and at least one light detector. The light source and the light detector can be separated by a distance. The sensor 120 can include any number of light sources or light detectors (e.g., two light sources and two detectors to make four measurements). The light source can be located or disposed with a product or material produced by the production system 110, such as in a conduit 115 of the production system. The light source can receive a signal from computing system 105 to cause the light source to operate and produce light of a particular wavelength in the product. Similarly, the optical receiver can be disposed within the product. The optical receiver can be or include a camera, hyperspectral sensor, hyperspectral receiver, for example. The optical receiver can be separated from the light source by a distance, and receive light produced by the receiver. The distance can be a path that the light travels along. In some implementations, the transmitter and receiver are disposed next to each other for reflectance measurements. The product can be disposed between the light source and the receiver, so the light received by the light source can be effected (e.g., scattered, reflected, refracted, emitted, absorbed) by the product before being sensed by the optical receiver. The light detector can generate a second signal, and provide the second signal to the computing system 105 indicating sensed wavelengths of light. Using the first and second signals, the spectroscopy module 190 can identify a type of the microbe present in the product, an amount of the microbe, or whether the microbe is dead or alive.

The sensor 120 can sweep through different wavelengths of light produced by the light source. For example, a broadband spectrum or select wavelengths can be produced by the light source and detected by the light detector to understand the material of interest. For example, the spectral-based sensing can be performed in ultraviolet (UV), visible, near-infrared (NIR), or infrared (IR) ranges. The module 190 can apply raman spectroscopy to determine the presence and growth of microorganisms in a sample or vessel. The spectral sensor 120 can be placed in a line 115 in a non-sanitary or sanitary configuration. The spectroscopy module 190 can receive a spectral measurement 185, indicating intensities or power measured by a light detector, for various wavelengths of light produced by the light source. The spectral measurement 185 can be intensities or power for a spectrum of wavelengths or for one or a set of wavelengths.

The spectroscopy module 190 can provide the spectral measurements 185 to the model 150 or 155 on the computing system 105, in the network, on a computer system, or on an external network. The spectroscopy module 190 can use the spectral measurements 185 to determine the presence of spectral peaks and the change in the spectrum to understand the presence and growth rate of microorganisms.

The module 190 can use the spectral measurements 185 to measure microbial metabolites as well as chemicals consumed and created by microorganisms. The module 190 can use the spectral measurements 185 to predict microbe growth and change. The module 190 can use the spectral measurements 185 to determine material change, quality identification, process measurement, fermentation, chemical reaction, and the transition from one state to another. The module 190 or the machine learning engine 145 can use the spectral measurements 185 and one or multiple models to analyze different aspects of the spectrum 185 as well as changes in the spectrum 185 for different organisms, microorganisms, viruses, the chemicals and compounds consumed and created by them, as well as the metabolites created by them. As an example, the module 190 can use the spectral measurement 185 to measure chlorophyll and phycocyanin absorption spectrums to understand algae growth. The module 190 can use the spectral measurement 185 to understand the presence and amount of different types of algae and other microorganisms in a vessel. The module 190 can use the spectral measurement 185 of materials flowing through a spectral-based sensor or directed at a surface where biofilm can develop.

The spectroscopy module 190 can feed the spectral measurement 185 to models 150 and/or 155 for microbe monitoring and predictive modeling or in place of genomic sequencing data for understanding microbe identification and quantification. The applications, techniques, deployment configurations, use of multiple sensors, and application and deployment of models described for impedance spectroscopy sensors 120 are also applicable to spectral sensing. Models and algorithms can be tuned to spectral sensor data 185 and can be tailored to the application, environment, sample, and process of interest.

In some implementations, the computing system 105 can be integrated or coupled with a scheduling or disinfection system that schedules cleanings or disinfections for the production system 110. The computing system 105 can transmit or send data to cause a cleaning to be scheduled for the production system 110 in the future using the predicted growth. For example, the computing system 105 can compare the identified microbe determined by the microbe identification model 150 to a list of dangerous or unwanted microbes. The computing system 105 can determine a match between the identified microbe and a microbe in the list. The list can further include a maximum quantity or concentration of the microbe. The computing system 105 can receive the threshold quantity of the microbe, and determine a time in the future at which the microbe population will grow to or past the threshold. Based on the identified time, the computing system 105 can communicate with the scheduling system to schedule cleaning or disinfection at a particular time in advance of the identified time.

The system 100 can include at least one electrode configuration that can be positioned in a variety of arrangements relative to the production system. For example, electrode configurations can include electrodes in direct contact with the liquid medium in the production system, as well as contactless electrode configurations mounted onto the outside of a pipe, conduit, or vessel 115 of the production system 110 without direct contact with the liquid. The contactless electrode configuration can allow for non-invasive impedance measurements through the wall of the pipe, conduit, or vessel, enabling impedance spectroscopy without risk of contamination to the product or material within the production system.

The electrode configurations can include various designs to optimize sensitivity and measurement capabilities. For example, the electrodes can be designed as interdigitated electrodes having multiple finger-like projections extending from at least two bus bars, with the fingers of one bus bar interleaved with the fingers of the other bus bar. The interdigitated electrode design can increase the effective surface area for measurement while maintaining a compact form factor, thereby improving sensitivity to changes in impedance caused by microbes or their metabolites in the production system. The interdigitated electrodes can be fabricated on a flexible substrate to conform to curved surfaces, or on a rigid substrate for placement in strategic locations within or around the production system.

The impedance spectroscopy module 190 can be configured to measure electrical parameters across multiple electrode configurations simultaneously or sequentially, and can compare measurements between the direct-contact and contactless electrode configurations to calibrate, validate, or enhance the accuracy of the impedance measurements 195. The module 190 can further apply different signal processing techniques to measurements from different electrode configurations to extract complementary information about the microbe populations in the production system 110.

The system 100 can implement various electrode configurations specifically designed for cylindrical geometries such as pipes, tubes, or cylindrical vessels in the production system 110. For contact-based measurements, the electrodes can be configured as circumferential ring electrodes that extend around the full circumference of the inner surface of a pipe, for example. These ring electrodes can be positioned at predetermined distances from each other along the axial direction of the pipe to create an electric field that passes through the liquid medium flowing within the pipe. The ring electrode configuration ensures uniform electric field distribution across the cross-section of the pipe, providing representative impedance measurements of the entire fluid volume passing through the measurement zone.

In contact-based examples, the electrodes can be configured as partial-circumference electrodes that cover only a segment of the pipe's inner circumference. These partial-circumference electrodes can be arranged in pairs or groups at various positions around the circumference to create multiple measurement paths through the liquid medium. This configuration can provide spatial resolution of impedance measurements within the pipe cross-section, potentially detecting microbial concentrations or characteristics that vary across the pipe diameter, such as those that might occur due to laminar flow patterns or biofilm formation on specific sections of the pipe wall.

For contactless impedance measurements, the system 100 can implement capacitively-coupled electrode arrays that wrap around the outer surface of the pipe without requiring direct contact with the liquid medium. The capacitive electrodes can be designed as flexible, conformable conductive sheets or meshes that can be securely wrapped around the pipe's exterior surface. The electrodes can be fabricated on flexible printed circuit boards (PCBs) that can be tightened around pipes of various diameters, ensuring close proximity to the pipe surface for optimal capacitive coupling. The capacitive electrodes can be arranged in multiple segments around the pipe circumference and along the pipe axis to enable three-dimensional mapping of impedance variations within the flowing medium.

The interdigitated electrode configuration can be specially adapted for cylindrical geometries by fabricating the electrodes on flexible substrates that conform to the curvature of the pipe. For example, the interdigitated electrodes can be arranged such that the fingers extend in the axial direction of the pipe, with multiple sets of interdigitated electrode pairs positioned around the circumference of the pipe. The interdigitated electrodes can also be arranged with the fingers extending circumferentially around the pipe, with multiple sets positioned along the axial direction of the pipe. These arrangements can allow for increased sensitivity to impedance changes in either the axial or circumferential direction, depending on the specific microbial monitoring requirements of the production system 110.

The impedance spectroscopy module 190 can implement differential measurement techniques using electrode pairs positioned at different locations along the pipe. For example, a first set of electrodes can be positioned upstream in the flow, with a second set positioned downstream. By comparing the impedance measurements between these electrode sets, the system 100 can detect changes in microbial populations as the liquid flows through the pipe section, potentially identifying growth rates or changes in microbial characteristics in real-time. The differential measurements can also help compensate for baseline variations in the liquid medium's conductivity or dielectric properties that are not related to microbial activity. Comparisons can also be done not in real time with a set time interval or dynamically between two time points based on a signal input or presence of desired material to be analyzed passing through the sensors.

The impedance spectroscopy module 190 can output a single conductivity reading or a scan of readings. The compute system 105 or machine learning module 145 can output a single conductivity reading, scan of readings, alert, command, or control signal.

The electrodes can be integrated with the pipe structure itself, such as by embedding conductive materials within the pipe wall during manufacturing or by creating specialized pipe sections with integrated electrode arrays that can be installed at strategic locations within the production system 110. These integrated electrode configurations can be designed to minimize disruption to flow patterns while maximizing sensitivity to microbial-induced impedance changes. The integrated design can also enhance durability and reduce the risk of contamination or electrode degradation in harsh production environments.

The system 100 can implement multilayer electrode configurations for enhanced sensitivity and specificity in cylindrical geometries. These configurations can include or consist of multiple layers of electrodes separated by insulating materials, with each layer optimized to detect impedance changes at different penetration depths into the liquid medium. By analyzing the impedance measurements from different electrode layers, the system 100 can distinguish between microbes distributed throughout the liquid and those concentrated near the pipe walls, such as in biofilm formations. This multilayer approach can also help discriminate between impedance changes caused by microbes and those resulting from other factors such as temperature variations or non-microbial particulates.

The sensor 120 can include at least one optical density measurement apparatus comprising a light-emitting diode (LED) and a photodiode mounted onto a pipe, conduit, or vessel of the production system 110. The LED and photodiode can be positioned to measure optical density through a liquid containing particles or microbial cells flowing through the production system. The optical density measurement can be used to determine concentration, growth rate, or other characteristics of microbes in the production system in real-time or near real-time.

The LED and photodiode can be oriented at various angles relative to each other to optimize detection sensitivity and specificity. For example, the LED and photodiode can be oriented at approximately 90 degrees to each other to measure scattered light, or at approximately 135 degrees to measure light scattered at wider angles, which can provide different information about the size, shape, and concentration of microbes in the production system. The angle between the LED and photodiode can be selected based on the specific microbe types being monitored and the characteristics of the production system.

The LED can be selected to emit light in wavelengths ranging from 600 to 900 nanometers, 900-3000 nm, 400 to 600 nm, 100 nm to 1 mm. Other ranges greater than or less than these ranges are also possible. Different wavelengths within this range can be selected based on the specific absorption or scattering properties of the target microbes or the liquid medium. Multiple LEDs with different wavelengths can be used to generate a spectral profile that can enhance identification capabilities of the system.

The optical density measurement apparatus can incorporate electronic circuits utilizing a chopper amplifier frequency that transforms the measurement signal to a higher frequency. This frequency transformation can effectively remove the influence of incident light and other low-frequency noise sources, improving the signal-to-noise ratio of the measurements. The chopper amplifier can modulate the LED at a specific frequency and synchronize the photodiode detection with this modulation, allowing the system to filter out ambient light and other interference that would otherwise reduce measurement accuracy. This technique enables the optical density measurements to be performed in environments with varying lighting conditions without compromising the accuracy or reliability of the microbial detection and characterization.

The system 100 can implement advanced optical density measurement configurations specifically designed for industrial pipe applications where liquids flow continuously through the production system 110. For example, the optical density measurement apparatus can be integrated into a specialized pipe section with optical windows or ports that allow light to pass through the flowing liquid while maintaining the structural and pressure integrity of the pipe. These optical windows can be constructed from materials such as sapphire, quartz, or specialized polymers that offer excellent optical transparency at the desired wavelengths while providing sufficient durability for industrial environments. The windows can be designed with self-cleaning geometries that minimize the accumulation of deposits that might otherwise interfere with optical measurements.

For larger diameter pipes, the system can implement a multi-path optical measurement configuration where multiple LED-photodiode pairs are positioned at different locations around the pipe circumference and along the pipe length. This configuration enables the system 100 (e.g., computing system 105) to measure optical density across different cross-sections of the flowing liquid, potentially detecting spatial variations in microbial concentrations or characteristics that might occur due to flow patterns, gravity effects, or local growth conditions. The measurements from multiple paths can be integrated by the computing system 105 to generate a three-dimensional profile of optical density throughout the pipe volume, providing more comprehensive monitoring than single-path measurements.

The system 100 can incorporate collimation optics for both the light source and detector to enhance measurement precision in pipe applications. For the LED, collimating lenses or apertures can focus the light into a narrow beam that passes through a specific region of the flowing liquid. For the photodiode, similar optics can ensure that only light traveling along the desired path is detected, reducing the influence of scattered light from particulates or bubbles outside the measurement zone. The collimation optics can be adjustable to allow optimization for different pipe diameters, liquid types, or specific monitoring requirements of the production system 110.

To accommodate variations in pipe diameter and installation constraints, the optical density measurement apparatus can be designed with adjustable mounting mechanisms that allow precise positioning of the LED and photodiode relative to the pipe. These mounting mechanisms can include articulated arms, sliding brackets, or rotational joints that enable fine adjustment of the angle between the LED and photodiode. The mounting system can also include stabilization features to minimize vibration-induced measurement variations in industrial environments with significant mechanical activity.

For monitoring nanobubbles used in cleaning and sanitizing processes, the system 100 can implement specialized optical density measurement techniques optimized for nanobubble detection and characterization. These techniques can include multi-angle scattering measurements where multiple photodiodes are positioned at different angles relative to the LED to capture light scattered by nanobubbles at various angles. The pattern and intensity of scattered light at different angles can provide information about nanobubble concentration, size distribution, and stability. For enhanced nanobubble characterization, the system 100 can implement dynamic light scattering techniques that analyze the temporal fluctuations in scattered light intensity caused by the Brownian motion of nanobubbles in the liquid.

The optical density measurement apparatus can incorporate wavelength-selective technologies to enhance specificity for particular microbes or nanobubbles. These technologies can include bandpass filters positioned in front of the LED, the photodiode, or both, to restrict the measurement to specific wavelength ranges where the target microbes or nanobubbles have distinctive optical properties. Alternatively, the system can implement dichroic mirrors or beam splitters that direct different wavelengths to separate photodiodes, enabling simultaneous multi-wavelength measurements. The wavelength-selective components can be chosen based on the specific absorption, reflection, or scattering spectra of the target microbes or nanobubbles in the production system 110.

For continuous online monitoring in industrial settings, the optical density measurement apparatus can implement automatic calibration mechanisms that periodically verify and adjust the measurement baseline. These mechanisms can include reference channels where light bypasses the liquid medium, providing a continuous reference for source intensity variations. The system can also include calibration routines that temporarily divert a portion of the flowing liquid through a reference cell with known optical properties. The automatic calibration can compensate for drift in the LED output, photodiode sensitivity, or optical component degradation over time, ensuring consistent and accurate measurements in long-term industrial deployments.

The system can implement pulsed-light measurement techniques for enhanced sensitivity and interference rejection in industrial environments. The LED can be driven with precisely timed pulses of varying duration, intensity, and frequency patterns, with the photodiode detection synchronized to these patterns. This synchronization, combined with advanced signal processing techniques such as lock-in amplification or correlation analysis, can extract the optical density signal from background noise even in challenging industrial environments with varying ambient light conditions, electromagnetic interference, or process-related optical disturbances.

For comprehensive monitoring of cleaning and sanitization processes using nanobubbles, the optical density measurement apparatus can implement time-resolved techniques that track changes in optical properties during and after nanobubble application. These techniques can monitor the generation, stability, and eventual collapse of nanobubbles in the system, providing insights into the effectiveness and coverage of the cleaning process. By correlating the optical measurements with other sensor data and production system parameters, the machine learning engine 145 can develop predictive models for optimal nanobubble-based cleaning schedules and protocols specific to the microbial populations present in the production system 110. Dissolved oxygen, oxygen reduction potential (ORP), gas sensors can be applied independently or in combination for this application.

The system 100 can implement differential optical density measurement techniques that compare measurements taken at different locations along the flow path to detect changes in microbial populations or nanobubble characteristics as the liquid moves through the production system 110. For example, optical measurements taken before and after a treatment section, heat exchanger, or reactor can provide direct evidence of the effectiveness of these processes in controlling microbial growth or maintaining nanobubble stability. The differential measurements can help isolate the effects of specific production system components on microbial populations, enabling more targeted interventions and optimizations. The system can identify concentration, size, characteristics of nanobubbels. The system can also identify particulate sizes, droplet sizes, concentration, mixture level, emulsification level, homogenization, etc.

For enhanced measurement capabilities in turbid or optically dense liquids common in industrial settings, the system can implement spatially resolved diffuse reflectance techniques. This approach utilizes multiple photodiodes positioned at different distances from the light source to measure how light is scattered and absorbed as it travels through the liquid. The spatial distribution of reflected light intensity can be analyzed to extract both absorption and scattering coefficients of the liquid, providing more complete information about both the concentration and physical properties of microbes and nanobubbles than traditional single-path optical density measurements.

The optical density measurement apparatus can incorporate temperature compensation mechanisms to maintain measurement accuracy across the wide temperature ranges often encountered in industrial production systems. These mechanisms can include temperature sensors integrated with the optical components, along with algorithms that adjust the measurement interpretation based on known temperature dependencies of the optical properties of the liquid medium, microbes, and nanobubbles. Additionally, the LED and photodiode assemblies can include thermal stabilization features such as heat sinks, thermal insulation, or active temperature control elements to minimize measurement drift due to temperature fluctuations.

For monitoring biofilm formation on pipe walls, the optical density measurement apparatus can implement specialized configurations that direct light at shallow angles relative to the pipe wall. This configuration enhances sensitivity to changes in the optical properties of the liquid-wall interface where biofilms typically develop. Multiple LED-photodiode pairs can be arranged around the pipe circumference to monitor different sections of the pipe wall, potentially detecting non-uniform biofilm development. This wall-focused optical monitoring can be particularly valuable for early detection of biofilm formation before it becomes extensive enough to affect bulk liquid measurements or impact production system performance.

In examples that include monitoring nanobubbles for cleaning and sanitization, the system 100 can utilize fluorescence-based techniques in addition to or instead of traditional optical density measurements. By incorporating LEDs that emit at excitation wavelengths for certain fluorescent dyes or naturally fluorescent compounds, and photodiodes with appropriate filters to detect the resulting fluorescence emission, the system can monitor the interaction between nanobubbles and microbes or biofilms. These interactions might include enhanced penetration of antimicrobial compounds into biofilms, physical disruption of microbial cells, or changes in metabolic activity following nanobubble exposure, all of which might be detectable through changes in fluorescence properties.

The optical density measurement apparatus can implement wavelength-scanning capabilities to generate spectral profiles of the flowing liquid. By sequentially activating LEDs of different wavelengths, or by utilizing broadband light sources combined with wavelength-selective detection, the system can measure optical density across a range of wavelengths. These spectral profiles can provide fingerprinting capabilities for different types of microbes or nanobubble populations, enhancing the specificity of the monitoring system. The machine learning engine 145 can be trained to recognize specific spectral patterns associated with different microbial species, growth phases, or nanobubble characteristics relevant to the production system 110.

The system 100 can include at least one radio frequency (RF) sensing apparatus comprising resonating coil elements tuned to RF frequencies ranging from 50 to 150 MHz, 75 to 150 MHz, 1 to 50 MHz, 1 kHz to 1 MHz, 150 MHz to 1 GHz, 10 kHz to 1 GHz. Other ranges can be greater than or less than these ranges. The RF sensing apparatus can be positioned around or adjacent to a conduit, pipe, or vessel of the production system 110 to non-invasively monitor microbe populations and characteristics without direct contact with the product or material within the production system.

The RF sensing apparatus can include measurement electronics such as a vector network analyzer or similar electronic circuit capable of extracting antenna parameters including, but not limited to, the resonant frequency, the matching or phase of the reflected signal, and the quality factor (Q) of the resonating coil elements. These parameters can be monitored continuously or at specified intervals to detect changes in the electromagnetic properties of the liquid or material passing through or contained within the RF coil's field.

The computing system 105 can be configured to monitor the RF parameters over time as they are influenced by the liquid or material passing through the RF coil. The presence, concentration, and characteristics of microbes in the liquid or material can affect the coupling of the tuned and matched coil, resulting in potential resonance shifts and matching differences. These changes can lead to measurable variations in the full width at half maximum (FWHM) of the resonance curve and other RF parameters that the system can analyze.

The microbe machine learning engine 145 can be trained to recognize patterns in the RF parameter changes that correlate with specific types, concentrations, or states of microbes. The engine 145 can integrate the RF sensing data with other sensor data, such as genetic information 140, impedance measurements 195, or optical measurements, to enhance the accuracy and specificity of microbe identification and growth prediction. The RF sensing apparatus can be particularly effective for detecting changes in microbe populations before they reach levels that would be detectable by other sensing methods, enabling earlier intervention and more precise control of the production system 110. The machine learning engine 145 can also be applied to measure presence, type, concentration of microbes, chemicals, contaminants, materials, characteristics of the sample being measured. The machine learning engine 145 can output a control, signal, or command for alert, logging, or action.

The RF sensing apparatus can utilize multiple resonating coil elements operating at different frequencies within the 50 to 150 MHz, 75 to 150 MHZ, 1 to 50 MHz, 1 kHz to 1 MHZ, 150 MHz to 1 GHz, 10 kHz to 1 GHz range (or other ranges greater than or less than these ranges) to provide a more comprehensive electromagnetic profile of the microbes in the production system. The multiple frequency measurements can be analyzed collectively by the microbe machine learning engine 145 to differentiate between different types of microbes or to separate the signals of microbes from other materials or contaminants in the production system 110.

The RF sensing apparatus can implement various coil configurations specifically designed for cylindrical geometries in the production system 110. For example, the RF coil can be configured as a solenoid coil that encircles the pipe, with multiple turns of conductive wire or trace wrapped around the pipe's exterior. The solenoid configuration generates a magnetic field primarily oriented along the axis of the pipe, with the field lines passing through the liquid medium flowing within the pipe. The number of turns, spacing between turns, and overall length of the solenoid can be optimized based on the pipe diameter, wall material, and specific frequency range to maximize sensitivity to microbial-induced changes in the electromagnetic properties of the liquid medium.

The RF sensing apparatus can utilize a saddle coil configuration that partially wraps around the pipe circumference. The saddle coil includes two arc segments positioned on opposite sides of the pipe, with connecting segments that run parallel to the pipe axis. This configuration generates a magnetic field oriented perpendicular to the pipe axis, providing different sensitivity patterns compared to the solenoid coil. Multiple saddle coils can be positioned around the pipe circumference to provide comprehensive coverage of the liquid medium. The saddle coil configuration can be advantageous when space constraints limit the installation of full circumferential coils, or when directional sensitivity is desired.

The RF sensing apparatus can implement a Helmholtz coil configuration, comprising two identical circular coils placed on either side of the pipe, with their centers aligned with the pipe axis and separated by a distance approximately equal to the coil radius. This configuration generates a relatively uniform magnetic field in the region between the coils, which can enhance the consistency of measurements across the pipe cross-section. The Helmholtz configuration can be effective for larger diameter pipes where penetration of the RF field through the entire liquid volume can be challenging with other coil designs.

For enhanced sensitivity and spatial resolution, the RF sensing apparatus can implement array configurations with multiple coil elements positioned at strategic locations around and along the pipe. These arrays can include combinations of different coil types, such as solenoid segments, saddle coils, or planar spiral coils, each tuned to specific frequencies within the 50-150 MHz, 75 to 150 MHz, 1 to 50 MHz, 1 kHz to 1 MHz, 150 MHz to 1 GHz, 10 kHz to 1 GHZ range (or other ranges greater than or less than these ranges). The coil array can enable differential measurements between adjacent coil elements, potentially detecting localized changes in microbial concentrations or characteristics. The system can implement MIMO (Multiple-Input Multiple-Output) techniques with the coil arrays, where different combinations of transmit and receive coils are activated in sequence to generate comprehensive electromagnetic profiles of the liquid medium.

The RF coils can be fabricated using various methods and materials optimized for cylindrical geometries. The coils can be manufactured as flexible printed circuit boards (PCBs) with conductive traces forming the coil pattern. These flexible PCBs can be wrapped around pipes of various diameters and secured in place, ensuring close proximity to the pipe surface for optimal coupling with the liquid medium. The coils can also be formed using rigid segments that clamp around the pipe, with precision alignment mechanisms to ensure optimal positioning relative to the pipe and other coil elements.

The RF sensing apparatus can include impedance matching networks specifically designed for cylindrical geometries. These matching networks can include adjustable components that can be tuned to compensate for variations in pipe diameter, wall thickness, material composition, and other factors that might affect the coupling between the RF coils and the liquid medium. The matching networks can be integrated into the coil assemblies or implemented as separate modules connected to the coils via transmission lines. The system can include automatic tuning mechanisms that periodically adjust the matching networks to maintain optimal sensitivity as environmental conditions or liquid medium characteristics change over time.

The RF coils can be designed with shielding configurations to minimize interference from external electromagnetic sources and to focus the RF fields on the liquid medium within the pipe. The shielding can include conductive materials positioned strategically around the coil assemblies to shape the field patterns and block external fields. The shielding configurations can be particularly important in production environments with multiple electrical machines, motors, or other equipment that might generate electromagnetic interference in the frequency range used by the RF sensing apparatus.

For pipes with non-conductive walls, the RF coils can be designed to maximize penetration of the RF fields through the pipe wall and into the liquid medium. For pipes with conductive walls or conductive coatings, the system can implement specialized coil configurations and frequencies that can induce currents in the conductive portions of the pipe, which in turn generate secondary fields that interact with the liquid medium. The specific coil design, operating frequency, and signal processing techniques can be selected based on the pipe material properties to optimize sensitivity to microbial-induced changes in the electromagnetic properties of the liquid medium.

Data from an optical receiver, electrode, antenna, or spectral receiver can be fed to compute system 105 or a model to identify material characteristics, microbe characteristics, type, concentration, chemistry, physical characteristics, quality, geometry, shape, size, composition, etc.

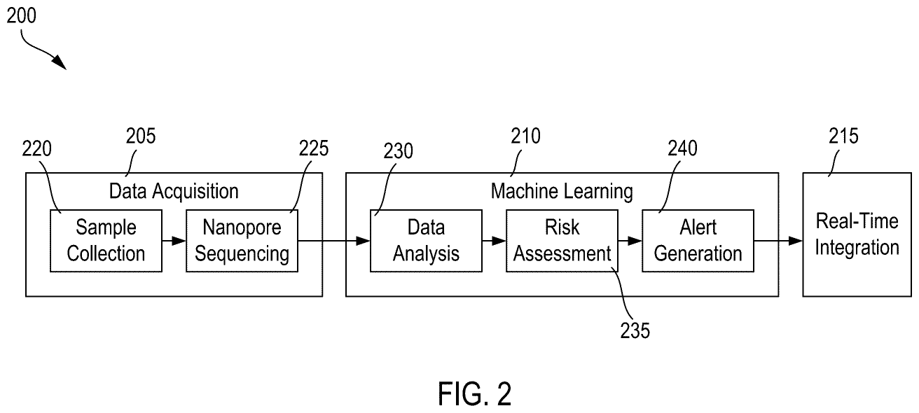

Referring now to FIG. 2, among others, an example method 200 of microbial sensing and predictive growth modeling is shown. The method 200 can include an ACT 205 of data acquisition. The method 200 can include an ACT 210 of machine learning. The method 200 can include an ACT 215 of real-time integration. The ACT 205 can include an ACT 220 of sample collection. The ACT 205 can include an ACT 225 of nanopore sequencing. The ACT 210 can include an ACT 230 of data analysis. The ACT 210 can include an ACT 235 of risk assessment. The ACT 210 can include an ACT 240 of alert generation. The production system 110, the computing system 105, the sequencing apparatus 135, the controller 125, or the client device 197 can perform at least a portion of the method 200.

At ACT 205, the method 200 can include acquiring data. Acquiring data can include collecting samples at ACT 220. For example, the sequencing apparatus 135 can collect samples of a medium, such as a liquid, fluid, powder, solid, plasma, or gas. The samples can be samples of a product or a material used to produce a product by the production system 110. The samples can be collected by a sequencing apparatus 135. At least one component or apparatus can retrieve or divert the sample via a port 175 from the production system 110 to the sequencing apparatus 135. The port 175 can provide continuous or periodic samples of the material to the sequencing apparatus 135. In some implementations, the method 200 can include air sampling, power sampling, surface swabbing, etc. to collect samples.

At ACT 225, the method 200 can include nanopore sequencing. The method 200 can include sequencing, by the sequencing apparatus 135, genetic information 140 present in the sample collected at ACT 220. The method 200 can include sequencing DNA or RNA. The method 200 can include sequencing genetic information 140 of microbes present in the sample collected at ACT 220. The method 200 can include determining an amount of concentration of certain genetic information 140, which can be indicative of a concentration or quantity of a microbe population.

At ACT 210, the method 200 can include performing machine learning. For example, the method 200 can include an ACT 230 of data analysis. The data analysis can include feeding data into models 150 or 155 of a microbe machine learning engine 145. For example, the engine 145 can feed genetic information 140 into the microbe identification model 150 to classify and quantify a microbe. The engine 145 can feed the classification and quantification of the microbe, along with the operational data 170, into the microbe growth model 155.

For example, the method 200 can include executing the microbe machine learning engine 145 to classify, quantify, or predict the growth of microbes in the production system 110. The machine learning engine 145 can identify the taxonomy of microbes present in the production system 110 using the genetic information 140. The machine learning engine 145 can include identifying patterns and relationships between specific microbial sequences, growth characteristics, population density, and potential impact on contamination. The machine learning engine 145 can determine or predict microbe population density in the production system 110 from the genetic information 140 based on the amount of RNA or DNA in a sample and the size of the sample. The machine learning engine 145 can determine population density in the production system 110 based on the population density in the sample and the size of the sample.

The method 200 can include performing a dynamics analysis of the microbes. For example, the machine learning engine 145 can predict or forecast the growth of the microbe into the future, based on the quantity of the microbe, the type of the microbe, and the environmental conditions in which the microbes are growing. Furthermore, the method 200 can include determining or predicting contamination impact of a microbe, e.g., spoilage, pathogenicity, etc.

The method 200 can include an ACT 235 of risk assessment. The computing system 105 can identify risk of contamination of a product by a microbe and trigger a preventative notification or control command 130. The computing system 105 can determine or assess risk that a microbe poses on consumer health, product yield, product quality, etc. The computing system 105 can compare a current concentration or quantity of a microbe to one or more thresholds. The computing system 105 can identify that the microbe is a dangerous microbe or unwanted microbe, and determine that the quantity of the microbe is less than a threshold. If the computing system 105 determines that the microbe is greater than a threshold, or will be greater than a threshold at a particular time in the future, at ACT 240, the computing system 105 can generate an alert. The alert can be an indication, recommendation, or a control command 130 to stop the production system 110, clean the production system 110, change an operating parameter of the production system 110, etc.

The method 200 can include determining microbial risk based on microbes identified and population densities of the identified microbes. The method 200 can include acquiring, by the computing system 105, growth characteristics of an environment where the microbes are located. For example, the method 200 can include acquiring, by the computing system 105, operational data 170, such as pH, temperature, nutrient availability, turbidity, flow rate, etc. of a line 115 or factory line. The method 200 can include receiving the operational data 170 from the controller 125, which can be coupled with various sensors that measure characteristics or environmental conditions of the production system 110.

The method 200 can include an ACT 215 of real-time integration. The real-time integration between the computing system 105, the client device 197, the sequencing apparatus 135, and/or the production system 110 can allow for predictive and proactive control, operation, cleaning, or sanitation of the production system 110. For example, the computing system 105, using the forecasted microbial growth, can determine when to sanitize or clean the production system 110, and display the determined sanitation or cleaning time in the graphical user interface 180 on the client device 197. Furthermore, the computing system 105 can, using the forecasted microbial growth, make product line operation adjustments for the production system 110. The computing system 105 can transmit control commands 130 to control the production system 110 and adjust how the production system 110 produces a product. This can simultaneously control growth of at least one microbe in the production system 110. Furthermore, the computing system 105 can generate information for display in the graphical user interface 180 on the client device 197. The computing system 105 can cause real-time warnings, alerts, or recommendations to be displayed within the client device 197.

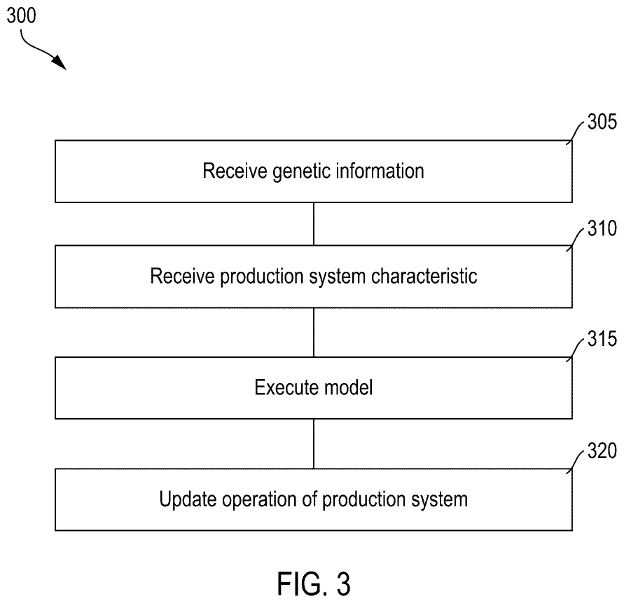

Referring now to FIG. 3, an example method 300 of microbial sensing and predictive growth modeling is shown. The method 300 can include an ACT 305 of receiving genetic information. The method 300 can include an ACT 310 of receiving a production system characteristic. The method 300 can include an ACT 315 of executing at least one model. The method 300 can include an ACT 320 of updating operation of a production system. The production system 110, the computing system 105, the sequencing apparatus 135, the controller 125, or the client device 197 can perform at least a portion of the method 300.

At ACT 305, the method 300 can include receiving, by the computing system 105, genetic information 140. The method 300 can include receiving the genetic information 140 from the sequencing apparatus 135. The method 300 can include receiving, by the sequencing apparatus 135, a sample from a line 115 of the production system 110. The method 300 can include receiving one sample, receiving samples at an interval, or continuously receiving samples. The samples can be received in-line from the production system 110. The samples can be samples of a medium, such as a liquid, fluid, powder, solid, plasma, or gas that includes at least one microbe.

At ACT 310, the method 300 can include receiving, by the computing system 105, production system characteristics. For example, the method 300 can include receiving, by the computing system 105, operational data 170 from the production system 110. The method 300 can include receiving, by the computing system 105, operational data 170 from the controller 125. Furthermore, the computing system 105 can receive characteristics from the client device 197 or from another device or server system. The operational data 170 can indicate or include environmental characteristics of the production system 110. The operational data 170 can include operating settings of the production system 110 (e.g., temperature setpoints, timer lengths, humidity setpoints, etc.) or sensor measurements of the production system 110 (e.g., in-line sensor measurements monitoring environmental parameters such as chemical composition, pH, salinity, temperature, chemistry, flow rate, turbidity, nutrient availability, biofilms, total dissolved solids (TDS), conductivity, RF data, image data, video data, optical data, alcohol level data, impedance spectroscopy, light spectroscopy, ultraviolet transmittance or transmission (UVT)).

At ACT 315, the method 300 can include executing, by the computing system 105, at least one model. For example, the method 300 can include executing a microbe identification model 150 or a microbe growth model 155. The method 300 can include executing a microbe machine learning engine 145 using the genetic information 140 received at ACT 305 and using the operational data 170 received at ACT 310. For example, the method 300 can include executing the microbe identification model 150 based on input data input into the model 150, such as the genetic information 140, an amount of the genetic information 140 in a sample, etc. The model 150 can output a taxonomy classification of the microbe and an amount of the microbe in the sample. The model 150 can output multiple taxonomy classification and microbe population quantities of multiple different types of microbes present in the sample. Furthermore, the method 300 can include executing a microbe growth model 155 to forecast or predict microbe growth in the production system 110 into the future. For example, the method 300 can include executing the microbe growth model 155 based on input data input into the model 150, such the classification of the microbes in the production system 110, the size or amount of microbes in the microbe population in the production system 110, and/or the operational data 170. The model 150 can output a forecasted quantity of the microbe population into one or multiple timesteps into the future.

At ACT 320, the method 300 can include updating, by the computing system 105, operation of the production system 110. For example, the method 300 can include updating, by the computing system 105, operation of the production system 110 using the classification of the microbe, the initial quantity of the microbe, and/or forecasted quantities of the microbe. The method 300 can include generating, by the computing system 105, at least one control command 130 that updates, changes, or adjusts the operation or performance of the production system 110 to produce a product. The control commands 130 can change heating temperature setpoints, change flowrate setpoints, change humidity setpoints, change a length of time to heat a material, change a length of time to cook a material, change a length of time to cool a material, etc. The method 300 can include executing, using various control commands 130, the microbe growth model 155 to identify how different control commands 130 will effect the growth of microbes in the production system 110. The method 300 can include executing, by the computing system 105, the model 155 multiple times or running an optimization algorithm to identify at least one control command 130 that slows the growth rate of the microbe (e.g., if the microbe is an undesirable microbe) or increases the growth rate of the microbe (e.g., if the microbe is classified as a desirable microbe).

Referring generally to FIGS. 1-3, at least one of the first electrode or the second electrode can be configured as a contactless electrode mounted on an exterior surface of the pipe without direct contact with the product carried within the pipe. The system 100 can include at least one of the first electrode or the second electrode configured as an interdigitated electrode comprising multiple finger-like projections extending from at least two bus bars, with the fingers of one bus bar interleaved with the fingers of another bus bar. The system 100 can include at least one of the first electrode or the second electrode is configured as a circumferential ring electrode that extends around a full or partial circumference of an inner or outer surface of the pipe.

A light source can be disposed on or adjacent to a pipe carrying a product produced by the production system 110, the light source can produce light, and the system 100 can include an optical receiver to dispose on or adjacent to the pipe at an angle relative to the light source. The angle can be between 90 and 180 degrees. The optical receiver can receive light after interaction with the product. The computing system 105 can analyze optical measurements from the optical received, and identify, based on the optical measurements, a type of the microbe, an amount of the microbe, whether the microbe is dead or alive, or a presence of nanobubbles in the product.

The light source can emit light in wavelengths ranging from 600 to 900 nanometers and can be modulated at a specific frequency. The optical receiver can be synchronized with the modulation to filter out ambient light and other interference.

The system 100 can include multiple light source and optical receiver pairs positioned at different locations around a circumference of the pipe and along a length of the pipe to generate a three-dimensional profile of optical density throughout a volume of the product within the pipe.

The computing system 105 can execute algorithms to detect and characterize nanobubbles used in cleaning and sanitizing processes in the production system 110.

The system 100 can include at least one radio frequency (RF) sensing apparatus comprising resonating coil elements tuned to RF frequencies ranging from 50 to 150 MHz, 75 to 150 MHz, 1 to 50 MHz, 1 kHz to 1 MHz, 150 MHz to 1 GHz, 10 kHz to 1 GHz (or other ranges greater than or less than these ranges) positioned around or adjacent to a pipe carrying a product produced by the production system. The computing system 105 can analyze antenna parameters of the resonating coil elements, and the antenna parameters can include at least one of a resonant frequency, a matching or phase of a reflected signal, or a quality factor. The computing system 105 can identify, based on changes in the antenna parameters over time, a type of the microbe, an amount of the microbe, or whether the microbe is dead or alive.

The resonating coil elements can be configured in at least one of a solenoid configuration encircling the pipe, a saddle coil configuration partially wrapping around the pipe circumference, or a Helmholtz coil configuration comprising two identical circular coils placed on opposite sides of the pipe.

The RF sensing apparatus can include multiple coil elements positioned at different locations around and along the pipe to enable differential measurements between adjacent coil elements for detecting localized changes in microbial concentrations or characteristics.

The one or more processors of the computing system 105 can implement multi-sensor fusion by integrating data from at least two different sensor types selected from genetic sequencing apparatus, impedance spectroscopy electrodes, optical density measurement apparatus, and radio frequency (RF) sensing apparatus to enhance accuracy of microbe detection and growth prediction.

The method 300 can includes receiving, by one or more processors coupled with memory, genetic information of a microbe in a production system, the genetic information sequenced from a sample taken from the production system. The method 300 can include receiving, by the one or more processors, sensor data from at least one sensor selected from impedance spectroscopy electrodes, optical density measurement apparatus, and radio frequency (RF) sensing apparatus. The method 300 can include executing, by the one or more processors, at least one model trained by machine learning or artificial intelligence using the genetic information and the sensor data to identify the microbe or determine a characteristic of the microbe. The method 300 can include updating, by the one or more processors, operation of the production system using the identity of the microbe or the characteristic of the microbe.

Some examples include one or more storage media storing instructions thereon, that, when executed by one or more processors, cause the one or more processors to perform operations. The operations can include receiving genetic information of a microbe in a production system, the genetic information sequenced from a sample taken from the production system; receiving sensor data from at least one of an impedance spectroscopy system with contactless electrodes mounted on an exterior of a pipe, an optical density measurement system configured to detect nanobubbles in a liquid flowing through a pipe, or a radio frequency (RF) sensing system with resonating coils mounted around a pipe. The operations can include executing at least one model trained by machine learning or artificial intelligence using the genetic information and the sensor data to identify the microbe or determine a characteristic of the microbe. The operations can include updating operation of the production system using the identity of the microbe or the characteristic of the microbe.

The system 100 can include a system for monitoring nanobubbles used in cleaning and sanitization processes. The system 100 can include one or more processors, coupled with memory, to receive optical measurements from an optical density measurement apparatus positioned on a pipe in a production system, the optical density measurement apparatus comprising a light source and a photodiode positioned at an angle relative to each other. The one or more processors can analyze the optical measurements to detect and characterize nanobubbles in a liquid flowing through the pipe. The one or more processors can determine an effectiveness of a cleaning or sanitization process using the nanobubbles based on the optical measurements. The one or more processors can update operation of the production system based on the determined effectiveness of the cleaning or sanitization process.

The optical density measurement apparatus can utilize multiple wavelengths of light to generate spectral profiles of the nanobubbles, and the one or more processors of the computing system 105 can execute machine learning or artificial intelligence models to correlate the spectral profiles with specific nanobubble characteristics relevant to the cleaning or sanitization process.

The system 100 can include a system for monitoring microbial growth in a production system, includes a multi-modal sensing apparatus. The apparatus can include a genetic sequencing apparatus to generate genetic information of a microbe from a sample taken from the production system; an impedance spectroscopy system with electrodes positioned on a pipe of the production system; and an optical density measurement system with light sources and photodiodes positioned on the pipe. The apparatus can include a radio frequency (RF) sensing system with resonating coils positioned around the pipe. The system can include one or more processors, coupled with memory, to receive and integrate data from the multi-modal sensing apparatus; execute at least one model trained by machine learning or artificial intelligence using the integrated data to identify the microbe and predict growth of the microbe in the production system and update operation of the production system based on the identified microbe and the predicted growth.

The system 100 can includes multiple electrodes disposed within or external to a pipe carrying a product produced by the production system, the electrodes can arranged in a configuration to enable impedance measurements across multiple paths through the product. One or more processors can apply electrical signals to at least a subset of the electrodes. The one or more processors can receive response signals from at least another subset of the electrodes. The one or more processors can process the response signals to generate impedance measurements. The one or more processors can identify, based on the impedance measurements, a type of the microbe, an amount of the microbe, or whether the microbe is dead or alive.

At least a portion of the plurality of electrodes can be configured as contactless electrodes mounted on an exterior surface of the pipe without direct contact with the product carried within the pipe.

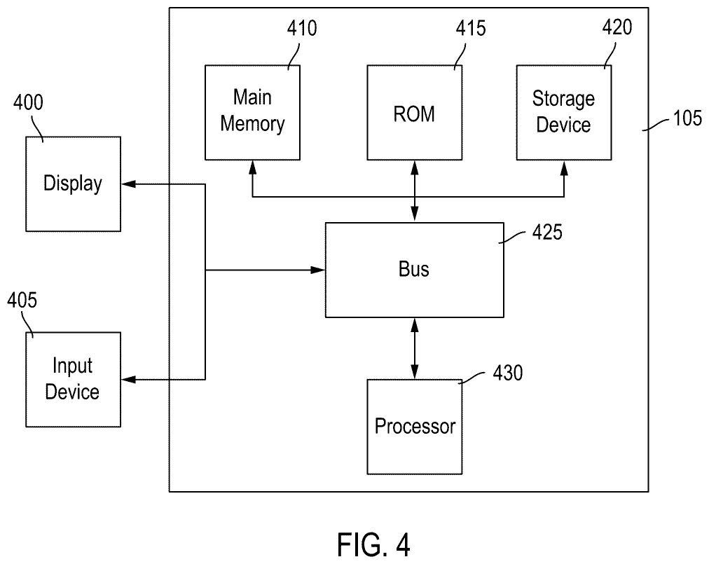

Referring now to FIG. 4, among others, an example block diagram of a computing system 105 is shown. The computing system 105 can include or be used to implement a data processing system or its components. The architecture depicted in FIG. 4 can be used to implement a component of the production system 110, the controller 125, the sequencing apparatus 135, the client device 197, or the computing system 105. The computing system 105 can include at least one bus 425 or other communication component for communicating information and at least one processor 430 or processing circuit coupled to the bus 425 for processing information. The computing system 105 can include one or more processors 430 or processing circuits coupled to the bus 425 for processing information. The computing system 105 can include at least one main memory 410, such as a random access memory (RAM) or other dynamic storage device, coupled to the bus 425 for storing information, and instructions to be executed by the processor 430. The main memory 410 can be used for storing information during execution of instructions by the processor 430. The computing system 105 can further include at least one read only memory (ROM) 415 or other static storage device coupled to the bus 425 for storing static information and instructions for the processor 430. A storage device 420, such as a solid state device, magnetic disk or optical disk, can be coupled to the bus 425 to persistently store information and instructions.

The computing system 105 can be coupled via the bus 425 to a display 400, such as a liquid crystal display, or active matrix display. The display 400 can display information to a user such as an operator, technician, or user of the production system. An input device 405, such as a keyboard or voice interface can be coupled to the bus 425 for communicating information and commands to the processor 430. The input device 405 can include a touch screen of the display 400. The input device 405 can include a cursor control, such as a mouse, a trackball, or cursor direction keys, for communicating direction information and command selections to the processor 430 and for controlling cursor movement on the display 400.

The processes, systems and methods described herein can be implemented by the computing system 105 in response to the processor 430 executing an arrangement of instructions contained in main memory 410. Such instructions can be read into main memory 410 from another computer-readable medium, such as the storage device 420. Execution of the arrangement of instructions contained in main memory 410 causes the computing system 105 to perform the illustrative processes described herein. One or more processors in a multi-processing arrangement can be employed to execute the instructions contained in main memory 410. Hard-wired circuitry can be used in place of or in combination with software instructions together with the systems and methods described herein. Systems and methods described herein are not limited to any specific combination of hardware circuitry and software.

Although an example computing system has been described in FIG. 4, the subject matter including the operations described in this specification can be implemented in other types of digital electronic circuitry, or in computer software, firmware, or hardware, including the structures disclosed in this specification and their structural equivalents, or in combinations of one or more of them.

Some of the description herein emphasizes the structural independence of the aspects of the system components or groupings of operations and responsibilities of these system components. Other groupings that execute similar overall operations are within the scope of the present application. Modules can be implemented in hardware or as computer instructions on a non-transient computer readable storage medium, and modules can be distributed across various hardware or computer based components.

The systems described above can provide multiple ones of any or each of those components and these components can be provided on either a standalone system or on multiple instantiation in a distributed system. In addition, the systems and methods described above can be provided as one or more computer-readable programs or executable instructions embodied on or in one or more articles of manufacture. The article of manufacture can be cloud storage, a hard disk, a CD-ROM, a flash memory card, a PROM, a RAM, a ROM, or a magnetic tape. In general, the computer-readable programs can be implemented in any programming language, such as LISP, PERL, C, C++, C #, PROLOG, or in any byte code language such as JAVA. The software programs or executable instructions can be stored on or in one or more articles of manufacture as object code.

Example and non-limiting module implementation elements include sensors providing any value determined herein, sensors providing any value that is a precursor to a value determined herein, datalink or network hardware including communication chips, oscillating crystals, communication links, cables, twisted pair wiring, coaxial wiring, shielded wiring, transmitters, receivers, or transceivers, logic circuits, hard-wired logic circuits, reconfigurable logic circuits in a particular non-transient state configured according to the module specification, any actuator including at least an electrical, hydraulic, or pneumatic actuator, a solenoid, an op-amp, analog control elements (springs, filters, integrators, adders, dividers, gain elements), or digital control elements.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatuses. Alternatively, or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices include cloud storage). The operations described in this specification can be implemented as operations performed by a data processing apparatus on data stored on one or more computer-readable storage devices or received from other sources.

The terms “computing device”, “component” or “data processing apparatus” or the like encompass various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, app, script, or code) can be written in any form of programming language, including compiled or interpreted languages, declarative or procedural languages, and can be deployed in any form, including as a stand-alone program or as a module, component, subroutine, object, or other unit suitable for use in a computing environment. A computer program can correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit). Devices suitable for storing computer program instructions and data can include non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

The subject matter described herein can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or a combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or embodiment, and references to “an implementation,” “some implementations,” “one implementation” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. Elements shown as integrally formed can be constructed of multiple parts or elements, the position of elements can be reversed or otherwise varied, and the nature or number of discrete elements or positions can be altered or varied. For example the system 100 can include the computing system 105 and components thereof, such as one or more processors 430. Other substitutions, modifications, changes and omissions can also be made in the design, operating conditions and arrangement of the disclosed elements and operations without departing from the scope of the present disclosure.

What is claimed is:

1. A system, comprising:
one or more processors, coupled with memory, to:
receive genetic information of a microbe in a production system, the genetic information sequenced from a sample taken from the production system;
execute at least one machine learning technique using the genetic information to identify the microbe or determine a characteristic of the microbe; and
update operation of the production system using the identity of the microbe or the characteristic of the microbe.

2. The system of claim 1, comprising:
a radio frequency sensing apparatus to sense radio frequency information of the sample;
wherein the one or more processors execute the machine learning technique using the sensed radio frequency information to identify the microbe or determine the characteristic of the microbe.

3. The system of claim 1, comprising:
the one or more processors to receive the genetic information from a rapid genetic sequencing apparatus located on-premises with the production system, the rapid genetic sequencing apparatus to sequence the genetic information of the microbe from the sample.

4. The system of claim 1, comprising:
the one or more processors to:
receive a characteristic of the production system used to control the production system to produce a product;
execute the machine learning technique using the genetic information and the characteristic of the production system to predict growth of the microbe in the production system; and
operate, using the predicted growth of the microbe in the production system, the production system to produce of the product and control the growth of the microbe in the production system.

5. The system of claim 1, comprising:
the one or more processors to:
receive an indication of a metabolite produced by the microbe in the production system, the indication determined through chemical or spectral sensing;
identify the microbe based on a type of the metabolite; and
determine that the microbe is alive based on the indication of the metabolite.

6. The system of claim 1, comprising:
at least one component to receive the sample from a conduit of the production system and provide the sample to a sequencing apparatus;
the one or more processors to:
continuously receive the genetic information from the sequencing apparatus; and
continuously identify, using the genetic information, the microbe and predict growth of the microbe in the production system.

7. The system of claim 1, comprising;
a first electrode to dispose within a product produced by the production system in a conduit of the production system;
a second electrode to dispose within the product in the conduit of the production system, the first electrode and the second electrode separated by a distance; and
the one or more processors to:
sweep a frequency of a first signal applied to the first electrode;
receive a second signal from the second electrode based on the first signal applied to the first electrode; and
identify, based on a change in amplitude or phase of the second signal relative to the first signal, a type of the microbe, an amount of the microbe, or whether the microbe is dead or alive.

8. The system of claim 1, comprising:
a light source to dispose within a product produced by the production system in a conduct of the production system, the light source to produce light in the product;
an optical receiver to dispose within the product to receive the light along a path; and
the one or more processors to:
determine a first signal indicating one or more wavelengths of light and their respective one or more amplitudes produced by the light source;
receive a second signal indicating the one or more wavelengths of light and their respective one or more amplitudes received by the optical receiver; and
identify, based on the first signal and the second signal, a type of the microbe, an amount of the microbe, or whether the microbe is dead or alive.

9. The system of claim 1, comprising:
the one or more processors to:
execute a first model trained by machine learning using the genetic information to identify the microbe; and
execute a second model trained by machine learning using the identified microbe and an operating characteristic of the production system to predict growth of the microbe in the production system.

10. The system of claim 1, comprising:
the one or more processors to:
determine that the microbe is classified as a dangerous microbe; and
update the operation of the production system to slow growth of the microbe responsive to the determination that the microbe is classified as the dangerous microbe.

11. The system of claim 1, comprising:
the one or more processors to:
determine that the microbe is a yeast for the production system to produce a product with; and
responsive to the determination that the production system produces the product with the yeast, update the operation of the production system to control growth of the yeast to a level.

12. The system of claim 1, comprising:
the one or more processors to:
identify a time that an amount of the microbe will increase to a threshold amount using the predicted growth of the microbe; and
schedule a cleaning or disinfection of the production system at or before the identified time.

13. The system of claim 1, comprising:
the one or more processors to:
receive first genetic information of the microbe sequenced from the sample taken from the production system;
determine a first amount of the microbe using the first genetic information;
receive second genetic information of the microbe sequenced from the sample taken from the production system after the sample is inoculated for a duration of time;
determine a second amount of the microbe using the second genetic information;
classify, using the first amount and the second amount, the microbe as dead or alive; and
execute the machine learning technique using the classification of the microbe.

14. A method, comprising:
receiving, by one or more processors, coupled with memory, genetic information of a microbe in a production system, the genetic information sequenced from a sample taken from the production system;
executing, by the one or more processors, a machine learning technique using the genetic information to identify the microbe or determine a characteristic of the microbe; and
updating, by the one or more processors, operation of the production system using the identity of the microbe or the characteristic of the microbe.

15. The method of claim 14, comprising:
receiving, by the one or more processors, the genetic information from a rapid genetic sequencing apparatus located on-premises with the production system, the rapid genetic sequencing apparatus to sequence the genetic information of the microbe from the sample.

16. The method of claim 14, comprising:
receiving, by the one or more processors, a characteristic of the production system used to control the production system to produce a product;
executing, by the one or more processors, the machine learning technique using the genetic information and the characteristic of the production system to predict growth of the microbe in the production system; and
operating, by the one or more processors, using the predicted growth of the microbe in the production system, the production system to produce of the product and control the growth of the microbe in the production system.

17. The method of claim 14, comprising:
receiving, by the one or more processors, an indication of a metabolite produced by the microbe in the production system, the indication determined through chemical or spectral sensing;
identifying, by the one or more processors, the microbe based on a type of the metabolite; and
determining, by the one or more processors, that the microbe is alive based on the indication of the metabolite.

18. The method of claim 14, comprising:
continuously receiving, by the one or more processors, the genetic information from a sequencing apparatus that receives samples from a conduit of the production system and sequences the genetic information; and
continuously identifying, by the one or more processors, using the genetic information, the microbe and predict growth of the microbe in the production system.

19. One or more storage media storing instructions thereon, that, when executed by one or more processors, cause the one or more processors to perform operations, comprising:
receiving genetic information of a microbe in a production system, the genetic information sequenced from a sample taken from the production system;
executing a machine learning technique using the genetic information to identify the microbe or determine a characteristic of the microbe; and
updating operation of the production system using the identity of the microbe or the characteristic of the microbe.

20. The one or more storage media of claim 19, the operations comprising:
receiving a characteristic of the production system used to control the production system to produce a product;
executing the at least one model trained by machine learning using the genetic information and the characteristic of the production system to predict growth of the microbe in the production system; and
operating, using the predicted growth of the microbe in the production system, the production system to produce of the product and control the growth of the microbe in the production system.
